(19) United States
(12) Patent Application Publication
(10) Pub. No.: US 2003/0125265 A1
(43) Pub. Date: Jul. 3, 2003

(54) Anti-estrogen receptor agents for chemotherapy

(76) Inventors: Mien-Chie Hung, Houston, TX (US); Yiu-Keung Lau, Williamsville, NY (US); Yong Wen, South San Francisco, CA (US)

Correspondence Address: Melissa L. Sistrunk, Fulbright & Jaworski L. L. P., 600 Congress Avenue, Suite 2400, Austin, TX, 78701, US

(21) Appl. No.: 10/142,115

(22) Filed: May 9, 2002

Related U.S. Application Data
(60) Provisional application No. 60/289,658, filed on May 9, 2001.

(51) Int. Cl. A61K 31/7048; A61K 31/353; A61K 31/12

(52) U.S. Cl. 514/27; 514/456; 514/680

(57) ABSTRACT

Methods and compositions regarding the prevention of ER-positive cancer and the treatment of ER-positive HER-2/neu-negative breast cancer are disclosed. Compositions exhibiting both tyrosine kinase inhibitor activity and anti-estrogen receptor activity are useful in the cancer treatment.

The present application claims priority to U.S. Provisional Patent Application No. 60/289,658, filed May 9, 2001.

### FIELD OF THE INVENTION

The present invention is directed to the field of tumor biology. Specifically, the present invention is directed to the prevention and/or treatment of cancer. More specifically, the present invention is directed to methods and compositions regarding tyrosine kinase inhibitors for the prevention, treatment, or prevention and treatment of cancer.

### BACKGROUND OF THE INVENTION

Aberrancies in expression of estrogen receptor (ER) has been associated with a variety of cancers, including breast cancer, endometrial cancer, cervical cancer and ovarian cancer. In ER positive breast cancer, positive receptor status is associated with favorable prognostic attributes including a lower rate of cell proliferation and histologic evidence of tumor differentiation. During the first several years following diagnosis, patients having ER-positive tumors tend to have a lower recurrence rate, but this is balanced by a higher recurrence rate in subsequent years, which results in an overall modest prognostic significance. A useful aspect to having ER positive cancer is in predicting response to hormonal therapy, both in the adjuvant setting and for advanced disease. Several therapies, including prevention and treatment, are available for ER positive cancers, although tamoxifen, a triphenyl derivative, is currently the most prevalent. Tamoxifen reduces proliferation of ER positive cancer cells through an estradiol antagonist mechanism, although the drug increases the risk for endometrial cancer, and, moreover, some patients develop resistance to it (for review, see Jordan, 1994).

The complexities attributed to cancer biology are in large part a result of the multitude of molecular and cellular phenotypes associated with it, even within a given tissue or organ. For example, there are many categories of breast cancer, such as those associated with ER status, progesterone receptor status, tyrosine kinase amplification, and so forth. In breast cancer having tyrosine kinase amplification, inhibitors of tyrosine kinases, such as emodin, are currently known to be useful in reducing proliferation of cells in vitro.

Emodin is a naturally occurring anthraquinone present in the roots and bark of numerous plants of the genus Rhamnus. Emodin has been reported to be a tyrosine kinase inhibitor that restricts the activity of p₅₆^{lck} kinase by preventing the binding of ATP in vitro (Jayasuriya et al., 1992). Emodin also can inhibit the growth of cancer cells, including lymphocytic leukemia (Kupchan et al., 1976), HL-60 human leukemia cells (Yeh et al., 1988), and ras-transformed human bronchial epithelial cells (Chan et al., 1993), by an unknown mechanism.

Emodin is particularly suited to treatment of HER-2/neu positive cancers. The neu gene (also known as HER-2/neu or c-erbB-2) encodes a 185-kDa transmembrane tyrosine kinase (p185^{neu}) with homology to epidermal growth factor receptor (Hung et al., 1986; Coussens et al., 1985; Schechter et al., 1984; Sanba et al., 1985; Yamamoto et al., 1986). Enhanced expression of neu is known to be involved in many human cancers, including non-small cell lung cancer (NSCLC) and has been shown to correlate with poor patient survival in NSCLC (Kern et al., 1990; Schneider et al., 1989; Weiner et al., 1990), and the gene is amplified in approximately 30% of primary breast cancers. Cellular and animal studies have shown that an increase in neu tyrosine kinase activity increases the expression of malignant phenotypes (Muller et al., 1988; Hudziak et al., 1987; Muthuswamy et al., 1994; Yu et al., 1991; Yu et al., 1993; Hung et al., 1989; Sistonen et al., 1989; Yu et al., 1994).

In U.S. Pat. No. 6,172,212, incorporated by reference herein in its entirety, emodin is shown to inhibit neu tyrosine kinase activity and preferentially represses the transformation ability and growth rate of neu-overexpressing breast cancer cells.

The delivery of emodin-like tyrosine kinase inhibitors to cancer cells is described for example, by Hung et al. in PCT/US97/01686, incorporated by reference herein in its entirety. Hung et al. have demonstrated that emodin and emodin-like compounds suppress the tyrosine kinase activity of human breast cancer cells, suppress their transforming ability, and induce their differentiation. Further, Hung et al. have found that emodin also suppresses tyrosine phosphorylation of neu in lung cancer cells and preferentially inhibits growth of these cells. Further, it appears that emodin is able to sensitize lung cancer cells that overexpress neu to the chemotherapeutic agents cisplatin, doxorubicin, and VP16 (See, e.g., PCT/US97/01686).

Although several references (see, for example, Reddy et al., 1992; Monti and Sinha, 1994; Di Domenico et al., 1996; Tesarik et al., 1999; Nakagawa et al., 2000) describe tyrosine kinase inhibitors such as genistein or RG-13022 for inhibiting cell proliferation in estrogen receptor (ER)-positive human breast carcinoma cell lines, there is no specific demonstration of their use for chemoprevention nor is the HER-2/neu phenotype in these cell lines defined. Furthermore, emodin and its derivatives are known for the suppression of growth of HER-2/neu-overexpressing breast cancer cell lines (see, for example, Zhang et al., 1995; Zhang et al., 1998; Zhang et al., 1999). Emodin has also been shown to sensitize HER-2/neu overexpressing chemoresistant non-small cell lung cancer (NSCLC) cells (Zhang and Hung, 1996) or breast cancer cells (Zhang et al., 1999) to chemotherapeutic drugs.

In contrast to these references, the methods and compositions of the present invention satisfy a need in the art for chemotherapeutic and chemopreventive measures against ER-positive HER-2/neu negative breast cancers.

Although some tyrosine kinase inhibitors are known to treat ER positive cancers (Reddy et al., 1992; Monti and Sinha, 1994; Di Domenico et al., 1996; Tesarik et al., 1999; Nakagawa et al., 2000), they are not known to be useful for chemoprevention of ER positive cancers. Given that the risk factors for developing breast cancer are known and there is a beneficial utility of preventing a recurrence of breast cancer, the present invention fulfills a need in the art to provide a prophylaxis for ER positive cancers. That is, additional therapies against breast cancer are needed, particularly given that some breast cancers become resistant to tamoxifen over time (for review, see Jordan, 1994).

Tyrosine kinase inhibitors, such as emodin, have been well suited for the treatment of HER-2/neu positive cancers, unrelated to the ER nature of the cancer, especially given the tyrosine kinase nature of HER-2/neu; however, they have not been utilized for the treatment of ER positive HER-2/neu negative cancers. The present invention satisfies a deficiency in the art related to a dual approach to combat cancers which are both HER-2/neu negative and ER positive through tyrosine kinase inhibitor compounds, such as emodin.

### SUMMARY OF THE INVENTION

The present invention regards methods and compositions directed to estrogen receptor positive (ER positive) cancers, and, in some preferred embodiments, to ER positive breast cancers. Such cancers include, for example: ovarian, endometrial, cervical, lung cancers, head and neck cancers, melanoma, meningiomas, thymomoas and lymphomas. In some specific embodiments, the invention relates to the prevention, treatment, or prevention and treatment of ER positive breast cancers.

In a preferred embodiment, the present invention regards prevention of the development or proliferation of an ER positive cancer cell, such as an ER positive breast cancer cell in an individual. The prevention utilizes a composition having anti-estrogen receptor activity, and in some particular embodiments such compositions also have tyrosine kinase inhibitor activity. The anti-estrogen receptor activity is associated with the same composition as the tyrosine kinase inhibitor activity and can comprise any means to affect the estrogen receptor such that it prevents transduction of the hormone estrogen signal and/or prevents an increase in estrogen in the cell or tissue, particularly in a cell or tissue in which the increase in estrogen would result in harmful effects. Thus, the anti-estrogen receptor activity includes a decrease in levels of estrogen receptor in the cell, an increase in its degradation, a downregulation of expression of the polynucleotide which encodes it, or a decrease in the halflife of a mRNA generated from a polynucleotide which encodes it.

In particular embodiments, the composition also comprises tyrosine kinase inhibitor activity, wherein the activity comprises reducing, impeding, obstructing, or otherwise interfering with, preferably in a deleterious manner, the activity of a tyrosine kinase. Such interference includes affecting any domain within the tyrosine kinase, such as a catalytic domain, a regulatory domain, an extracellular domain, and so forth. The inhibition can be, for example, the result of the tyrosine kinase inhibitor affecting the protein structure, including removal or modification of amino acid residues, increasing the degradation of the polypeptide, blocking access to a particular domain, such as the catalytic domain, affecting expression levels of the polynucleotide which encodes it, or decreasing the half-life of the mRNA which is expressed from the polynucleotide which encodes it. Specific examples of tyrosine kinase inhibitors are well known in the art, including emodin, genistein, and RG13022.

In other embodiments, the present invention addresses treatment of an ER positive HER-2/neu negative breast cancer cell in an individual. The treatment comprises contacting the cell with a composition having both tyrosine kinase inhibitor activity and anti-estrogen receptor activity.

In a preferred embodiment, the prevention of the development or proliferation of an ER positive cell with a composition having anti-ER activity or the treatment of the ER positive HER-2/neu negative cell with a composition having both tyrosine kinase inhibitor activity and anti-estrogen receptor activity occurs in an individual having a risk of developing breast cancer or an individual which has already received treatment for the breast cancer. The previous breast cancer therapy could comprise surgery, chemotherapy, radiation, or a combination thereof. In a specific embodiment, the individual having already received treatment for the ER positive breast cancer has the cancer in remission. A skilled artisan recognizes that breast cancer which recurs is not necessarily of the same type as was seen with the original occurrence, and therefore, in a specific embodiment all individuals having had breast cancer, regardless of the original etiology, are candidates for prevention and treatment with the compositions and methods described herein.

Furthermore, an individual who is at risk for developing breast cancer or having a recurrence of breast cancer is particularly well-suited to receive therapy with the methods and compositions described herein. A skilled artisan recognizes the multiple risk factors for an individual to develop breast cancer, including lifestyle and environmental factors, genetic factors, and so forth. Moreover, one skilled in the art recognizes histopathologies and specific mutations which are indicative of an increased risk for developing breast cancer, particularly with premalignant lesions.

Thus, in an object of the present invention there is a method of preventing development or proliferation of one or more estrogen receptor positive cancer cells in an individual comprising administering to the individual a composition having anti-estrogen receptor activity. In a specific embodiment, the composition also has tyrosine kinase inhibitor activity. In another specific embodiment, the anti-estrogen receptor activity comprises reducing estrogen receptor levels in the cell. In an additional specific embodiment, the anti-estrogen receptor activity comprises modification of the estrogen receptor in the individual. In specific embodiments, the modification comprises degradation of the estrogen receptor or downregulation of expression of an estrogen receptor polynucleotide. In a preferred embodiment, the composition is emodin. In a specific embodiment, the composition is emodin, genistein, or RG13022. In another specific embodiment, the cell is in vivo. In a further specific embodiment, the cell is in an animal. In an additional specific embodiment, the animal is a human. In a specific embodiment, the human is at an increased risk for developing breast cancer, such as having a typical ductal hyperplasia, a typical lobular hyperplasia, a typical epithelial hyperplasia, unfolded lobules, usual ductal hyperplasia, ductal carcinoma in situ, and lobular carcinoma in situ, a defective BRCA1 polynucleotide, a defective BRCA2 polynucleotide, an A908G mutation of an estrogen receptor alpha nucleic acid sequence, a breast cancer family history, or a radial scar.

In another object of the present invention, there is a method of treating an estrogen receptor positive and HER-2/neu negative breast cancer cell comprising contacting the cell with a composition comprising tyrosine kinase inhibitor activity and anti-estrogen receptor activity. In a specific embodiment, the composition is emodin. In a further specific embodiment, the composition is emodin, genistein, or RG13022. In another specific embodiment, the cell is in vivo. In a further specific embodiment, the cell is in an animal. In an additional specific embodiment, the animal is a human.

In accordance with the methods of the present invention, the contacting of the cell with the composition is concomitant with or subsequent to administration of a breast cancer therapy to said human. In a specific embodiment, the breast cancer therapy is radiation, surgery, chemotherapy, biological therapy, immunotherapy, or gene therapy. In an additional specific embodiment, the surgery is lumpectomy or a mastectomy of at least one breast of the individual. In another specific embodiment, the chemotherapy comprises an anthracycline, a taxane, an alkylating agent, a fluoropyrimidine, an antimetabolite, a vinca alkaloid, a platinum, or a combination thereof. In a specific embodiment, the anthracycline is doxorubicin, epirubicin, liposomal doxorubicin, or mitoxantrone. In another specific embodiment, the taxane is paclitaxel or docetaxel. In a further specific embodiment, the alkylating agent is cyclophosphamide. In an additional specific embodiment, the fluoropyrimidine is capecitabine 40 or 5-fluorouracil. In another specific embodiment, the antimetabolite is methotrexate. In a further specific embodiiment, the vinca alkaloid is vinorelbine 41, vinblastine, or vincristine. In another specific embodiment, the platinum is carboplatin or cisplatin. In an additional specific embodiment, the chemotherapy is gemcitabine, mitomycin C, or herceptin.

In an additional object of the present invention, there is a method of screening for a compound comprising tyrosine kinase inhibitor activity and anti-estrogen receptor activity, comprising contacting an estrogen receptor positive and HER-2/neu negative breast cancer cell with a candidate substance; and assaying the candidate substance for tyrosine kinase inhibitor activity and anti-estrogen receptor activity in said cell. In a specific embodiment, the cell is in an animal, and wherein the animal is assayed for said tyrosine kinase inhibitor activity and said anti-estrogen receptor activity. In a specific embodiment, the method further comprises placing the compound in a pharmacologically acceptable excipient. In a specific embodiment, the method further comprises using the compound in the pharmacologically acceptable excipient to treat an animal having estrogen receptor positive HER-2/neu negative breast cancer. In a specific embodiment, the animal is a human.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B. Emodin mediated chemopreventive activity of breast tumor development in MMTV-neu transgenic mice (1A) and in MMTV-v-Ha-ras transgenic mice (1B).

FIG. 2A and FIG. 2B. Emodin inhibits estrogen-induced DNA synthesis, and Rb hyperphosphorylation in MCF breast cancer cells.

FIG. 3A, FIG. 3B, FIG. 3C, and FIG. 3D. Depletion of estrogen receptor protein in estrogen receptor positive cells by emodin.

FIG. 4A, FIG. 4B, and FIG. 4C. Emodin-enhanced estrogen receptor protein degradation.

FIG. 5. Involvement of proteasome pathway in estrogen-induced estrogen receptor degradation.

FIG. 6. Effect of different protease inhibitors on emodin-induced estrogen receptor protein degradation.

FIG. 7A and FIG. 7B. Enhanced association of hsp90 and estrogen receptor protein in MCF-7 cells after incubation with emodin.

FIG. 8. An illustration of how emodin may induce estrogen receptor degradation.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

I. The Present Invention

The present invention is directed to the use of emodin and like anti-ER compounds to prevent ER positive cancers. A second aspect of the invention is the use of emodin and like anti-ER compounds to prevent and/or treat ER positive cancers. A further aspect of the invention is the prevention or treatment of ER positive cancers that are also HER-2/neu negative.

In the present invention, emodin prevented breast tumor development in a transgenic mice model. In a specific embodiment, other emodin-like ER depletion compounds prevent breast tumor development. In a specific embodiment, the mechanism for the chemoprevention activity is mediated through downregulation of the estrogen receptor in addition to suppression of tyrosine kinase activity. As shown herein, emodin downregulates estrogen receptor. This phenomenon results in suppression of estrogen, including mitogenic activity. Estrogen is a key factor to stimulate estrogen receptor (ER) positive breast cancer growth, and anti-estrogens such as tamoxifen and 4-hydroxytamoxifen have been used as therapeutic and chemopreventive agents for breast cancer patients. Thus, the current invention teaches that tyrosine kinase inhibitors such as emodin serve as a chemoprevention agent and are useful for treatment of ER-positive cancer patients. Tyrosine kinases such as EGF receptor, Her-2/neu, src, abl, etc., have been known to induce human cancer, and tyrosine kinase inhibitors have been used as anti-cancer agents to target tyrosine kinase-activated cancer. However, the present invention teaches anti-estrogen receptor activity in addition to tyrosine kinase inhibitor activity as: 1) cancer prevention for the ER-positive cancer patients (in particular, breast cancer patients); and 2) as an anti-cancer agent for cancer therapy. The novelty of the present invention is that a single agent can be used to inhibit tyrosine kinase activity and also deplete estrogen receptor. Thus, tyrosine kinase inhibitors such as emodin serve as broad and more useful therapeutic and/or preventive agents for ER-positive cancer patients than antiestrogens such as tamoxifen.

Specifically, the inventors show that emodin was useful in treating an estrogen receptor-positive breast cancer cell line, MCF-7. Emodin inhibited estrogen-induced mitogenic activity and Rb phosphorylation in a very similar way as antiestrogens, tamoxifen and 4-hydroxytamoxifen. Suppression of estrogen-induced functions by emodin accompanied depletion of the estrogen receptor, and this decrease in receptor protein was due to enhanced degradation. To examine the mechanism involved, inhibitors of the lysosomal, calpains, and proteasome proteolytic pathways were used. Only proteasome inhibitors blocked emodin-induced depletion of the estrogen receptor, which indicates the involvement of a proteasome pathway. Given that hsp90 plays a role in protein degradation and can form a complex with the estrogen receptor, it was examined whether emodin could affect this complex formation. Emodin treatment resulted in a marked increase in the complex formation, suggesting that normal dissociation of hsp90 from estrogen receptor was disrupted and led to receptor degradation. Taken together, the results presented herein indicate that tyrosine kinase(s) is involved in the regulation of estrogen receptor, and emodin can deplete estrogen receptor through the proteasome pathway, thereby blocking estrogen-induced biological function. Thus, this implicates tyrosine kinase inhibitors as broader chemopreventive agents than the traditional agents, such as tamoxifen.

Furthermore, a skilled artisan recognizes based on the Examples provided herein that emodin can be used as a chemopreventive agent that differs from the conventional antiestrogens, such as tamoxifen, which competes with estrogen for binding with the receptor. Instead, emodin targets the estrogen receptor by enhancing the receptor degradation. It provides another novel approach to treat and prevent estrogen-responsive breast cancer. In addition, emodin is also a tyrosine kinase inhibitor, thus, it serves as dual function agent for ER-positive cancer.

II. Breast Cancer and Breast Cancer Chemoprevention

Breast cancer is a major health problem for women, particularly in Western cultures. Approximately 1 in 8 women will be diagnosed with breast cancer in their lifetime. Breast cancer is commonly treated by various combinations of surgery, radiation therapy, chemotherapy, and hormone therapy. Prognosis and selection of therapy may be influenced by the age and menopausal status of the patient, stage of the disease, histologic and nuclear grade of the primary tumor, estrogen-receptor (ER) and progesterone-receptor (PR) status, measures of proliferative capacity, and HER2/neu gene amplification (Simpson et al., 2000). Certain rare inherited mutations such as those associated with BRCA1 and BRCA2 genes appear to predispose women to develop breast cancer. Breast cancer is classified into a variety of histologic types, some of which have prognostic importance. For example, favorable histologic types include mucinous, medullary, and tubular carcinoma (Rosen et al., 1991).

Predisposing factors include hormone replacement therapy of the individual. Hormone replacement therapy for postmenopausal women is a double-edged sword: the benefits seen with the administration of estrogen are outweighed by the risks of developing breast cancer.

The genotype of an individual also is a predisposing factor for breast cancer. Approximately 5% to 10% of all women with breast cancer may have a germ-line mutation of the genes BRCA1 and BRCA2 (Blackwood and Weber, 1998). Specific mutations of BRCA1 and BRCA2 are more prevalent in women of Jewish descent (Offit et al., 1996). For women with BRCA1 and BRCA2 mutations, the estimated lifetime risk of developing breast cancer is 40% to 85%, and male carriers of BRCA2 mutations are also at increased risk for breast cancer (The Breast Cancer Linkage Consortium, 1999). Carriers of the mutations having a history of breast cancer are at an increased risk of up to 5% per year (Frank et al., 1998). An increased risk of ovarian cancer also is associated with mutations in either BRCA1 or BRCA2, in addition to an increased risk of other primary cancers (The Breast Cancer Linkage Consortium, 1999; Miki et al., 1994; Ford et al., 1999).

One approach to combat the disease which has been used in recent years, particularly in patients who have already been treated for breast cancer or are at high risk, is chemoprevention using drugs. Women who are at an increased risk for developing the disease include those with a premalignant lesion, such as a biopsy-confirmed diagnosis of a typical epithelial hyperplasia (Osborne and Borgen, 1992), ductal carcinoma in situ (DCIS) (Schwartz et al., 1999), radial scar (Jacobs et al., 1999) and lobular carcinoma in situ (LCIS) (Osborne and Hoda, 1999). Furthermore, women are at high risk for the disease if they carry a mutation in the BRCA1 and BRCA2 genes (Marcus et al., 1996). The term “premalignant lesion” as used herein is defined as a collection of cells in a breast with histopathological characteristics which suggest at least one of the cells has an increased risk of becoming breast cancer. A skilled artisan recognizes that the most important premalignant lesions recognized today include unfolded lobules (UL; other names: blunt duct adenosis, columnar alteration of lobules), usual ductal hyperplasia (UDH; other names: proliferative disease without atypia, epitheliosis, papillomatosis, benign proliferative disease), a typical ductal hyperplasia (ADH), a typical lobular hyperplasia (ALH), ductal carcinoma in situ (DCIS), and lobular carcinoma in situ (LCIS). Other lesions which may have premalignant potential include intraductal papillomas, sclerosisng adenosis, and fibroadenomas (especially a typical fibroadenomas). In a specific embodiment, the collection of cells is a lump, tumor, mass, bump, bulge, swelling, and the like. Other terms in the art which are interchangeable with “premalignant lesion” include premalignant hyperplasia, premalignant neoplasia, and the like.

Women having a breast cancer family history are at increased risk for the disease. The term “breast cancer family history” as used herein refers to a relative of biological ancestry having any form of breast cancer, wherein the relative may be a great-grandmother, a mother, a sister, an aunt, a great aunt, or a cousin.

Premalignant lesions of the breast are very common, and they are being diagnosed more frequently due to increasing public awareness and screening mammography. They are currently defined by their histological features and their prognosis is imprecisely estimated based on indirect epidemiological evidence (Page and Dupont, 1993). While lesions within specific categories look alike histologically, there must be underlying biological differences causing a subset to progress to IBC. Studies identifying biological prognostic factors in premalignant disease are beginning to emerge (see discussions in Page and Jensen, 1994; Page, 1995; Page et al., 1998; Lakhani, 1999). The histopathological characteristics and anatomic markers associated with premalignant lesions are well known in the art (Cardiff et al., 1977; Bocker, 1997; Page and Dupont, 1990a, 1990b; Stoll, 1999; Lishman and Lakhani, 1999, each of which are incorporated by reference herein in their entirety).

The current line of attack for these high-risk patients includes administration of antiestrogens. The antiestrogen tamoxifen has been shown in more than one clinical trial to be useful in reducing the incidence of ER positive, but not ER negative, breast cancer (Early Breast Cancer Trialists Collaborative Group, 1998; Fisher et al., 1998), although other clinical trials have contradicted these findings (Veronisi et al., 1998; Powles et al., 1998). Moreover, deleterious side effects accompanied therapy with tamoxifen, including an increased incidence of endometrial cancer and some increases in the rate of pulmonary embolism, deep vein thrombosis, stroke, and development of cataracts.

Another therapy for chemoprevention includes the selective estrogen receptor modulators (SERMs) (Clemens et al., 1983), particularly raloxifene. In trials, raloxifene reduced the risk of ER-positive but not ER-negative breast tumors, and also did not cause any noted deleterious effects on the uterus (Cummings et al., 1999). Current investigation into alternative SERMs is ongoing.

In addition to the SERMs, natural compounds and their analogs are being investigated for potentially providing chemoprevention for breast cancer. Retinoids such as the synthetic vitamin A analog N-(4-hydroxyphenyl) retinamide (fenretinide) has shown in preclinical experimental studies (Moon et al., 1979) and clinical trials (Veronesi et al,. 1999) to have antitumor effects, but only for premenopausal woman. Also, Bradlow et al. (1995) have demonstrated that indole-3-carbinol, which induces the enzyme P450A1 that controls the formation of a metabolite that presumably blocks proliferation of mammary cells, is chemopreventive. Although no adverse effects are currently known for indole-3-carbinol, these studies are in early stages.

III. Tyrosine Kinase Inhibitors

Although the present invention is preferably directed to anti-estrogen receptor activity compositions for the prevention, treatment, or prevention and treatment of ER positive cancers, in particular embodiments the compositions also comprise tyrosine kinase activity. Tyrosine kinases are associated with many regulatory cellular processes, including oncogenesis. A multitude of tyrosine kinases are associated with human cancer, including the overexpression or amplification of HER-2/neu in breast cancer, EGFR in glioblastoma, Src in colon and breast cancer, and Rsc/Sky in breast cancer. A variety of approaches have been utilized for the development of inhibitors to tyrosine kinases. Specifically, much effort has been directed to developing inhibitors against one of the domains in tyrosine kinases, such as the catalytic domain, the adapter domain, the signaling domain, and a transmembrane domain or extracellular binding domain (where applicable). To date, the most successful agents target the Mg-ATP complex binding site of the catalytic domain of the enzyme, although there has been some success for agents which target other domains. For instance, herceptin, which is a monoclonal antibody against the extracellular domain of Her-2/neu (Baselga et al., 1996, Stebbing et al., 2000) has proven successful for Her-2 positive breast cancer therapy.

Libraries of compounds have been screened for kinase inhibitor activities, and in recent years combinatorial libraries have been useful in the identification of beneficial compounds. High-throughput assays have been utilized for screening protein kinase inhibitors, including the scintillation proximity assay (Braunwalder et al., 1996), the fluorescence polarization assay (Seethala and Menzel, 1997), and the heterogeneous time-resolved dissociation-enhanced fluorescence technology (Braunwalder et al., 1996). Furthermore, computational chemistry has been used increasingly for development of protein kinase inhibitors, particularly with the recent increase in the number of available crystal structures and with progress achieved in structure-based drug design (for review, see Al-Obeidi and Lam, 2000).

Peptide substrates for numerous protein tyrosine kinases have also been identified (Lam et al., 1995; Songyang et al., 1995, Lou et al., 1996; Wu et al., 1997, 1998; Schmitz et al., 1996). Potent inhibitors have been developed for protein kinase A based on modifying the components of a particular peptide substrate (Feramisco and Krebs, 1978; Walsh and Glass, 1991), and similar approaches have been utilized for protein tyrosine kinases (Wu et al., 1996; Lou et al., 1997; Alfaro-Lopez et al., 1998; Fry et al., 1994; Niu and Lawrence, 1997a, b; Walsh and Glass, 1991; Petrakis and Nagabhushan, 1987; Burke et al., 1993; Yuan et al., 1990).

Other substances for inhibition of protein tyrosine kinases include small molecule kinase catalytic domain inhibitors, some of which are in clinical trials (for review, see Al-Obeidi and Lam, 2000). Many are natural products and their derivatives, such as flavones and isoflavones, which are competitive inhibitors for ATP, and many are isolated from fungal species, such as Clitocyte clavips (Cassinelli et al., 2000) and Nocardiopsis species (Kase et al., 1997; Ruggeri et al., 1999). Classes of small molecule kinase catalytic domain inhibitors include quinazolines, pyridopyrimidines and related heterocyles, phenylamin-pyrimidines, benzylidene malonitrile (tyrphostins and their analoges), and indoles and oxindoles (for review, see Levitt and Kory, 1999; and Al Obeidi and Lam, 2000).

In addition to inhibitors of protein tyrosine kinases directed against the catalytic domain, some compounds target other domains, such as the extracellular domain of receptor protein tyrosine kinases (as with herceptin (Baselga et al., (2000) and references therein) against HER2) and the SH₂ domain (as with AP22408 (Shakespeare et al., 2000) or compound 9 (Lee and Lawrence, 2000) against Src).

IV. Combination Treatments

In order to increase the effectiveness of an anti-estrogen receptor or anti-estrogen receptor and tyrosine kinase inhibitor for cancer, it may be desirable to combine these compositions with other agents effective in the treatment of hyperproliferative disease, such as anti-cancer agents. An “anti-cancer” agent is capable of negatively affecting cancer in a subject, for example, by killing cancer cells,-inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the life span of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that anti-estrogen receptor tyrosine kinase inhibitor gene therapy could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

These can be, for example, agents that directly cross-link DNA, agents that intercalate into DNA, and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

1. Antibiotics

a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m²in persons having received mediastinal irradiation. Alternatively, 30 mg/m² on each of 3 consecutive days, repeated every 4 wk. Exemplary doses may be 10 mg/m², 20 Mg/m², 30 mg/m², 50 mg/m², 100 mg/m², 150 mg/m², 175 mg/m², 200 mg/m², 225 mg/m², 250 mg/m², 275 mg/M², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DNA-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 Mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 Mg/m², 400 mg/m², 425 mg/m², 450 mg/m², 475 mg/m², 500 mg/m². Of course, all of these dosages are exemplary, and any dosage in-between these points is also expected to be of use in the invention.

c Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways.

Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors which fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 Mg/m², 175 Mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. It is freely soluble in water.

Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

Bleomycin may be given by the intramuscular, intravenous, or subcutaneous routes.

2. Miscellaneous Agents

a. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m2, 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

In certain aspects of the current invention cisplatin is used in combination with emodin or emodin-like compounds in the treatment of non-small cell lung carcinoma. It is clear, however, that the combination of cisplatin and emodin and or emodin-like compounds could be used for the treatment of any other neu-mediated cancer.

b. VP16

VP16 is also know as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/M2 or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

c Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

3. Plant Alkaloids

a. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

b. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisolone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisolone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisolone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/ m², 0.15 mg/m², 0.2 mg/m², 0.25 mg/m² can be given as a constant intravenous infusion. Of course, all of these dosages are exemplary, and any dosage in-between these points is also expected to be of use in the invention.

c Vinblastine

When cells are incubated with vinblastine, dissolution of the microtubules occurs. Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours.

Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine for use will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/ m², 0.5 mg/ m², 1.0 mg/ m², 1.2 mg/ m², 1.4 mg/ m², 1.5 mg/ m², 2.0 mg/ m², 2.5 mg/ m², 5.0 mg/ m², 6 mg/ m², 8 mg/ m², 9 mg/ m², 10 mg/ m², 20 mg/ m², can be given. Of course, all of these dosages are exemplary, and any dosage in-between these points is also expected to be of use in the invention.

4. Alkylating Agents

a. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisolone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/ m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/ m² 40 mg/m² 50 mg/m² 60 mg/ m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject

b. Melphalan

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of 2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al, 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

c. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporated herein as a reference, for details on doses for administration.

d. Chlorambucil

Chlorambucil (also known as leukeran) was first synthesized by Everett et al. (1953). It is a bifunctional alkylating agent of the nitrogen mustard type that has been found active against selected human neoplastic diseases. Chlorambucil is known chemically as 4[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remingtons Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

e. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

f. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours.

The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-anti-estrogen receptor tyrosine kinase inhibitor gene therapy. The general approach for combined therapy is discussed below. Generally, the tumor cell must bear some marker that is amenable to targeting, i.e., is not present on the majority of other cells. Many tumor markers exist, and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as administration of an anti-estrogen receptor form or an anti-estrogen receptor and tyrosine kinase inhibitor form. Delivery of an anti-estrogen receptor tyrosine kinase inhibitor in conjuction with a vector encoding one of the following gene products will have a combined anti-hyperproliferative effect on target tissues. Alternatively, a single vector encoding more than one gene may be used. A variety of proteins, in conjunction with administration of an anti-estrogen receptor form or an anti-estrogen receptor and tyrosine kinase inhibitor, are encompassed within the invention, some of which are described below. In particular embodiments, the gene products are themselves also anti-estrogen receptor or anti-estrogen receptor and tyrosine kinase inhibitor gene products.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occuring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p₁₆^{B}, p19, anti-estrogen receptor tyrosine kinase inhibitor, and p₂₇^{KIP1}. The p16^{INK4} gene maps to a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et aL, 1994; Okamoto et al., 1994; Nobori et al., 1994; Orlow et al., 1994; Arap et al., 1995). Restoration of wild-type p₁₆^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p¹⁶ fusions, anti-estrogen receptor tyrosine kinase inhibitor/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

4. Definitions and Techniques Affecting Anti-ER and Anti-ER and Tyrosine Kinase Inhibitor Gene Products and Genes

a. Anti-ER and Anti-ER Tyrosine Kinase Inhibitor Gene Products and Genes

As used herein, the terms “anti-ER or anti-ER tyrosine kinase inhibitor gene product” and “anti-ER or anti-ER tyrosine kinase inhibitor” refer to proteins having amino acid sequences which are substantially identical to the native anti-estrogen receptor tyrosine kinase inhibitor or which are biologically active in that they are capable of cross-reacting with anti- anti-ER or anti-ER tyrosine kinase inhibitor antibody raised against an anti-ER compound or an anti-ER tyrosine kinase inhibitor, respectively. “Anti-ER or anti-ER tyrosine kinase inhibitor gene product” and “anti-ER or anti-ER tyrosine kinase inhibitor” refer to proteins having amino acid sequences which are substantially identical to the native anti-ER or anti-ER tyrosine kinase inhibitor amino acid sequence, respectively, and which are biologically active in that they are capable of binding to ETS binding sites or cross-reacting with anti- anti-ER or anti-ER tyrosine kinase inhibitor antibody raised against anti-ER or anti-ER tyrosine kinase inhibitor, respectively. Such sequences are disclosed, for example, in Macleod et al., (1992). The term “anti-ER or anti-ER tyrosine kinase inhibitor gene product” also includes analogs of anti-ER or anti-ER tyrosine kinase inhibitor molecules, respectively, which exhibit at least some biological activity in common with native anti-ER or anti-ER tyrosine kinase inhibitor, respectively. Furthermore, those skilled in the art of mutagenesis will appreciate that other analogs, as yet undisclosed or undiscovered, may be used to construct anti-ER or anti-ER tyrosine kinase inhibitor analogs, respectively.

The term “mutant form of anti-ER or anti-ER tyrosine kinase inhibitor” refers to any DNA sequence that is substantially identical to a DNA sequence encoding an anti-ER or anti-ER tyrosine kinase inhibitor gene product, respectively, as defined above. The term also refers to RNA, or antisense sequences compatible with such DNA sequences. An “anti-ER or anti-ER tyrosine kinase inhibitor gene” may also comprise any combination of associated control sequences.

The term “substantially identical”, when used to define either an anti-ER or anti-ER tyrosine kinase inhibitor amino acid sequence or an anti-ER or anti-ER tyrosine kinase inhibitor gene nucleic acid sequence, means that a particular subject sequence, for example, a mutant sequence, varies from the sequence of natural anti-ER or anti-ER tyrosine kinase inhibitor by one or more substitutions, deletions, or additions, the net effect of which is to retain at least some biological activity of the anti-ER or anti-ER tyrosine kinase inhibitor protein, respectively. Alternatively, DNA analog sequences are “substantially identical” to specific DNA sequences disclosed herein if: (a) the DNA analog sequence is derived from coding regions of the natural anti-ER or anti-ER tyrosine kinase inhibitor gene; or (b) the DNA analog sequence is capable of hybridization of DNA sequences of (a) under moderately stringent conditions and which encode biologically active anti-ER or anti-ER tyrosine kinase inhibitor; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequence.

b. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps.

c. Nucleic Acid Sequences

In certain embodiments, the invention concerns the use of anti-estrogen receptor tyrosine kinase inhibitor nucleic acids, genes and gene products, such as the anti-estrogen receptor tyrosine kinase inhibitor that includes a sequence which is essentially that of the known anti-estrogen receptor tyrosine kinase inhibitor gene, or the corresponding protein. The term “a sequence essentially as anti-estrogen receptor tyrosine kinase inhibitor” means that the sequence substantially corresponds to a portion of the anti-estrogen receptor tyrosine kinase inhibitor gene and has relatively few bases or amino acids (whether DNA or protein) which are not identical to those of anti-estrogen receptor tyrosine kinase inhibitor (or a biologically functional equivalent thereof, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of anti-estrogen receptor tyrosine kinase inhibitor will be sequences which are “essentially the same”.

Anti-ER or anti-ER tyrosine kinase inhibitor nucleic acids which have functionally equivalent codons are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glutamic Acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which may, for example, include various non-coding sequences flanking either of the 5′ or 3′ portions of the coding region or may include various internal sequences, i.e., introns, which are known to occur within genes.

The present invention also encompasses the use of DNA segments which are complementary, or essentially complementary, to the sequences set forth in the specification. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment in question under relatively stringent conditions such as those described herein.

d. Biologically Functional Equivalents

As mentioned above, modification and changes may be made in the structure of anti-ER or anti-ER tyrosine kinase inhibitor and still obtain a molecule having like or otherwise desirable characteristics. For example, certain amino acids may be substituted for other amino acids in a protein structure without appreciable loss of interactive binding capacity with structures such as, for example, a gene which encodes anti-ER or anti-ER tyrosine kinase inhibitor, respectively. Since it is the interactive capacity and nature of a protein that defines that protein's biological functional activity, certain amino acid sequence substitutions can be made in a protein sequence (or, of course, its underlying DNA coding sequence) and nevertheless obtain a protein with like or even countervailing properties (e.g., antagonistic v. agonistic). It is thus contemplated by the inventors that various changes may be made in the sequence of the anti-estrogen receptor tyrosine kinase inhibitor proteins or peptides (or underlying DNA) without appreciable loss of their desired biological utility or activity.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged.

Amino acid substitutions, such as those which might be employed in modifying anti-estrogen receptor tyrosine kinase inhibitor are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ^{±}2 is preferred, those which are within ^{±}1 are particularly preferred, and those within ^{±}0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

5. Additional Examples of Genes for Combination Therapy

A skilled artisan recognizes genes and gene products which would be useful for combination therapy with an anti-estrogen receptor or anti-estrogen receptor and tyrosine kinase inhibitor for the prevention, treatment, or prevention and treatment of an ER positive cell, and in particular embodiments an ER positive and HER-2/neu negative cell. In other specific embodiments, the cell is an ER positive breast cancer cell.

One example for combination therapy includes mini-ElA constructs that can be used for tumor suppression, as described in U.S. Pat. No. 6,197,754, incorporated by reference herein in its entirety or the adenoviral E1A constructs provided in U.S. Pat. Nos. 5,651,964; 5,643,567; 5,641,484, and 5,814,315, each of which is incorporated by reference herein in its entirety. Other examples of gene products for combination therapy with the anti-ER or anti-ER tyrosine kinase inhbitor compounds provided herein includes mutant forms of p21^{Cip1/WAF1}. In a specific embodiment, the mutant forms of p21^{Cip1/WAF1} inhibit proliferation of the cell.

6. In vivo Delivery and Treatment Protocols

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the neu-suppressing gene products, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of anti-estrogen receptor tyrosine kinase inhibitor will be preferred, there is no reason why other control sequences could not be employed, so long as they are compatible with the genotype of the cell being treated. Thus, one may mention other useful promoters by way of example, including, e.g., an SV40 early promoter, a long terminal repeat promoter from retrovirus, an actin promoter, a heat shock promoter, a metallothionein promoter, and the like.

For introduction of the nucleic acid encoding the mutant form of anti-estrogen receptor tyrosine kinase inhibitor, it is proposed that one will desire to preferably employ a vector construct that will deliver the desired gene to the affected cells. This will, of course, generally require that the construct be delivered to the targeted tumor cells, for example, breast, genital, or lung tumor cells. It is proposed that this may be achieved most preferably by introduction of the desired gene through the use of a viral or non-viral vectors to carry the anti-estrogen receptor tyrosine kinase inhibitor sequences to efficiently transfect the tumor, or pretumorous tissue. This infection may be achieved preferably by liposomal delivery but may also be via adenoviral, a retroviral, a vaccinia viral vector or adeno-associated virus.

These vectors have been successfully used to deliver desired sequences to cells and tend to have a high infection efficiency.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

e. Liposomal Transfection

Thus, an expression construct encoding a polypeptide of interest, or the polypeptide of interest itself, may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

The present invention also provides particularly useful methods for introducing anti-estrogen receptor or anti-estrogen receptor and tyrosine kinase inhibitor gene products into cells. One method of in vivo gene transfer which can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appears to be mainly spleen macrophages and liver Kupffer cells. Intravenous injection of liposomes/DNA complexes can lead to the uptake of DNA by these cellular sites, and result in the expression of a gene product encoded in the DNA (Nicolau, 1982).

The inventors contemplate that anti-estrogen receptor or anti-estrogen receptor and tyrosine kinase inhibitor gene products can be introduced into cells using liposome-mediated gene transfer. It is proposed that such constructs can be coupled with liposomes and directly introduced via a catheter, as described by Nabel et al. (1990). By employing these methods, the anti-estrogen receptor or anti-estrogen receptor and tyrosine kinase inhibitor gene products can be expressed efficiently at a specific site in vivo, not just the liver and spleen cells which are accessible via intravenous injection. Therefore, this invention also encompasses compositions of DNA constructs encoding an anti-estrogen receptor or anti-estrogen receptor and tyrosine kinase inhibitor gene product formulated as a DNA/liposome complex and methods of using such constructs.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3.beta.[N-(N′N′-dimethylaminoethane)-carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al., 1987, Nabel et al., 1990, and Gao et al., 1991. In a specific embodiment, the liposomes comprise DC-Chol. More particularly, the inventors the liposomes comprise DC-Chol and DOPE which have been prepared following the teaching of Gao et al. (1991) in the manner described in the Preferred Embodiments Section. The inventors also anticipate utility for liposomes comprised of DOTMA, such as those which arc available commercially under the trademark Lipofectin™, from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allows liposome-mediated transfer of DNA complex, for example, the liver and the spleen. In order to allow transfection of DNA into cells which are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding a nucleic acid encoding an anti-estrogen receptor tyrosine kinase inhibitor.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is anticipated to have utility, it is anticipated that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

In a specific embodiment, one employs the smallest region needed to enhance retention of anti-estrogen receptor tyrosine kinase inhibitor in the nucleus of a cell so that one is not introducing unnecessary DNA into cells which receive an anti-estrogen receptor or anti-estrogen receptor and tyrosine kinase inhibitor gene construct. Techniques well known to those of skill in the art, such as the use of restriction enzymes, will allow for the generation of small regions of anti-estrogen receptor tyrosine kinase inhibitor. The ability of these regions to inhibit neu can easily be determined by the assays reported in the Examples.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

In a further embodiment of the invention, the expression construct may be entrapped in a liposome. Liposomes are vesicular structures characterized by a phospholipid bilayer membrane and/or an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and/or entrap water and/or dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and/or expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and/or promote cell entry of liposome-encapsulated DNA (Kaneda et al., 1989). In other embodiments, the liposome may be complexed and/or employed in conjunction with nuclear non-histone chromosomal proteins (HMG-1) (Kato et al., 1991). In yet further embodiments, the liposome may be complexed and/or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

f. Adenovirus

Another method for in vivo delivery involves the use of an adenovirus vector.

“Adenovirus expression vector” is meant to include those constructs containing adenovirus sequences sufficient to (a) support packaging of the construct and (b) to express an polynucleotide that has been cloned therein.

Adenovirus is a particularly suitable gene transfer vector because of its midsized genome, ease of manipulation, high titer, wide arget-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 m.mu. is particularly efficient during the late phase of infection, and all the mRNA's issued from this promoter possess a 5′-tripartite leader (TL) sequence which makes them preferred mRNA's for translation.

In some cases, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A particular method of introducing the mutant form of anti-estrogen receptor tyrosine kinase inhibitor to an animal is to introduce a replication-deficient adenovirus containing the nucleic acid encoding the mutant form of anti-estrogen receptor tyrosine kinase inhibitor. The replication-deficient construct made by E1B and E3 deletion also avoids the viral reproduction inside the cell and transfer to other cells and infection of other people, which means the viral infection activity is shut down after it infects the target cell. The nucleic acid encoding the mutant form of anti-estrogen receptor tyrosine kinase inhibitor is still expressed inside the cells. Also, unlike retrovirus, which can only infect proliferating cells, adenovirus is able to transfer the nucleic acid encoding the mutant form of anti-estrogen receptor tyrosine kinase inhibitor into both proliferating and non-proliferating cells. Further, the extrachromosomal location of adenovirus in the infected cells decreases the chance of cellular oncogene activation within the treated animal.

Introduction of the adenovirus containing the neu-suppressing gene product gene into a suitable host is typically done by injecting the virus contained in a buffer.

The nature of the adenovirus vector is not believed to be crucial to the successful practice of the invention. Of course, as discussed above, it is advantageous if the adenovirus vector is replication defective, or at least conditionally defective, The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration in to the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1992; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotatic inoculation into the brain (Le Gal La Salle et al., 1993).

g. Retroviruses

The retroviruses are a group of single-stranded RNA viruses characterized by an ability to convert their RNA to double-stranded DNA to infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed y components is constructed (Mann et al., 1983). When a recombinant plasmid containing a human cDNA, together with the retroviral LTR and ψ sequences is introduced into this cell line (by calcium phosphate precipitation for example), the ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact ψ sequence from the recombinant virus inserts upstream from the gag, pol, env sequence integrated in the host cell genome. However, neu packaging cell lines are now available that should greatly decrease the likelihood of recombination (Markowitz et al., 1988; Hersdorffer et al., 1990).

One limitation to the use of retrovirus vectors in vivo is the limited ability to produce retroviral vector titers greater than 10⁶ infections U/mL. Titers 10- to 1,000-fold higher are necessary for many in vivo applications.

Several properties of the retrovirus have limited its use in lung cancer treatment (Stratford-Perricaudet and Perricaudet, 1992; (i) Infection by retrovirus depends on host cell division. In human cancer, very few mitotic cells can be found in tumor lesions. (ii) The integration of retrovirus into the host genome may cause adverse effects on target cells, because malignant cells are high in genetic instability. (iii) Retrovirus infection is often limited by a certain host range. (iv) Retrovirus has been associated with many malignancies in both mammals and vertebrates. (v) The titer of retrovirus, in general, is 100- to 1,000-fold lower than that of adenovirus.

h. Other Viral Vectors as Expression Constructs

Other viral vectors may be employed as expression constructs in the present invention. Vectors derived from viruses such as vaccinia virus (Ridgeway, 1988; Baichwal and Sugden, 1988; Coupar et al., 1988) adeno-associated virus (AAV) (Ridgeway, 1988; Baichwal and Sugden, 1988; Hermonat and Muzycska, 1984) and herpes viruses may be employed. They offer several attractive features for various mammalian cells (Friedmann, 1989; Ridgeway, 1988; Baichwal and Sugden, 1988; Coupar et al., 1988; Horwich et al., 1990).

With the recognition of defective hepatitis B viruses, neu insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Cultures media containing high titers of the recombinant virus were used to infect primary duckling hepatocytes. Stable CAT gene expression was detected for at least 24 days after transfection (Chang et al., 1991).

i. Other non-viral vectors

In order to effect expression of sense or antisense gene constructs, the expression construct must be delivered into a cell. This delivery may be accomplished in vitro, as in laboratory procedures for transforming cells lines, or in vivo or ex vivo (see below), as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984). direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al, 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells, in vitro, and then the return of the modified cells back into an animal. This may involve the surgical removal of tissue/organs from an animal or the primary culture of cells and tissues. Anderson et al., and U.S. Pat. No. 5,399,346, incorporated herein in its entirety, disclose ex vivo therapeutic methods.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1 beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

TABLE 2
ONCOGENES

| | | | |
|---|---|---|---|
| Gene | Source | Human Disease | Function |

Growth Factors¹

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma | PDGF B |
| | virus | |

Receptor Tyrosine Kinases^{1,2}

| | | | |
|---|---|---|---|
| ERBB/HER | Avian | Amplified, deleted | EGF/TGFα/ |
| | erythroblastosis | squamous cell | amphiregulin/ |
| | virus; ALV | cancer; | hetacellulin |
| | promoter | glioblastoma | receptor |
| | insertion; | | |
| | amplified | | |
| | human tumors | | |
| ERBB-2/NEU/HER-2 | Transfected from rat | Amplified breast, | Regulated by NDF/ |
| | Glioblatoms | ovarian, gastric | heregulin and |
| | | cancers | EGF- |
| | | | related factors |
| FMS | SM feline sarcoma | | CSF-1 receptor |
| | virus | | |
| KIT | HZ feline sarcoma | | MGF/Steel receptor |
| | virus | | hematopoieis |
| TRK | Transfection from | | NGF (nerve growth |
| | human colon | | factor) receptor |
| | cancer | | |
| MET | Transfection from | | Scatter factor/HGF |
| | human | | receptor |
| | osteosarcoma | | |
| RET | Translocations and | Sporadic thyroid | Orphan receptor Tyr |
| | point mutations | cancer; | kinase |
| | | familial medullary | |
| | | thyroid cancer; | |
| | | multiple endocrine | |
| | | neoplasias 2A and | |
| | | 2B | |
| ROS | URII avian sarcoma | | Orphan receptor Tyr |
| | Virus | | kinase |
| PDGF receptor | Translocation | Chronic | TEL(ETS-like |
| | | myclomonocytic | transcription |
| | | leukemia | factor)/ |
| | | | PDGF receptor |
| | | | gene |
| | | | fusion |
| TGF-βreceptor | | Colon carcinoma | |
| | | mismatch mutation | |
| | | target | |

NONRECEPTOR TYROSINE KINASES¹

| | | | |
|---|---|---|---|
| ABI. | Abelson Mul.V | Chronic | Interact with RB, |
| | | myelogenous | RNA |
| | | leukemia | polymerase, CRK, |
| | | translocation | CBL |
| | | with BCR | |
| FPS/FES | Avian Fujinami | | |
| | SV; GA | | |
| | FeSV | | |
| LCK | Mul.V (murine | | Src family; T cell |
| | leukemia | | signaling; interacts |
| | virus) promoter | | CD4/CD8 T cells |
| | insertion | | |
| SRC | Avian Rous | | Membrane- |
| | sarcoma | | associated Tyr |
| | Virus | | kinase with |
| | | | signaling function; |
| | | | activated by |
| | | | receptor kinases |
| YES | Avian Y73 virus | | Src family; |
| | | | signaling |

SER/THR PROTEIN KINASES¹

| | | |
|---|---|---|
| AKT | AKT8 murine | Regulated by |
| | retrovirus | PI(3)K?; |
| | | regulate 70-kd S6 |
| | | k? |
| MOS | Maloney murine SV | GVBD; cystostatic |
| | | factor; MAP |
| | | kinase |
| | | kinase |
| PIM-1 | Promoter insertion | |
| | Mouse | |
| RAF/MIL | 3611 murine SV; | Signaling in RAS |
| | MH2 | pathway |
| | avian SV | |

MISCELLANEOUS CELL SURFACE¹

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic |
| | | | binding; |
| | | | intracellular |
| | | | interacts with |
| | | | catenins |
| PTC/NBCCS | Tumor suppressor | Nevoid basal cell | 12 transmembrane |
| | and | cancer | domain; signals |
| | Drosophilia | syndrome (Gorline | through Gli |
| | homology | syndrome) | homogue |
| | | | CI to antagonize |
| | | | hedgehog pathway |
| TAN-1 Notch | Translocation | T-ALI. | Signaling? |
| homologue | | | |

MISCELLANEOUS SIGNALING^{1,3}

| | | | |
|---|---|---|---|
| BCL-2 | Translocation | B-cell lymphoma | Apoptosis |
| CBL | Mu Cas NS-1 V | | Tyrosine- |
| | | | phosphorylated |
| | | | RING |
| | | | finger interact Ab1 |
| CRK | CT1010 ASV | | Adapted SH2/SH3 |
| | | | interact Ab1 |
| DPC4 | Tumor suppressor | Pancreatic cancer | TGF-β-related |
| | | | signaling |
| | | | pathway |
| MAS | Transfection and | | Possible angiotensin |
| | Tumorigenicity | | receptor |
| NCK | | | Adaptor SH2/SH3 |

GUANINE NUCLEOTIDE EXCHANGERS AND BINDING
PROTEINS^{3,4}

| | | | |
|---|---|---|---|
| BCR | | Translocated with | Exchanger; protein |
| | | ABL | kinase |
| | | in CML | |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N- | HaRat SV; Ki | Point mutations in | Signal cascade |
| RAS | RaSV; | many | |
| | Balb-MoMuSV; | human tumors | |
| | Transfection | | |
| VAV | Transfection | | S112/S113; |
| | | | exchanger |

NUCLEAR PROTEINS AND TRANSCRIPTION FACTORS^{1,5-9}

| | | | |
|---|---|---|---|
| BRCA1 | Heritable suppressor | Mammary | Localization |
| | | cancer/ovarian | unsettled |
| | | cancer | |
| BRCA2 | Heritable suppressor | Mammary cancer | Function unknown |
| ERBA | Avian | | thyroid hormone |
| | erythroblastosis | | receptor |
| | Virus | | (transcription) |
| ETS | Avian E26 virus | | DNA binding |
| EVII | MuLV promotor | AML | Transcription factor |
| | Insertion | | |
| FOS | FBI/FBR murine | | 1 transcription |
| | osteosarcoma | | factor |
| | viruses | | with c-JUN |
| GLI | Amplified glioma | Glioma | Zinc finger; cubitus |
| | | | interruptus |
| | | | homologue |
| | | | is in hedgehog |
| | | | signaling pathway; |
| | | | inhibitory link |
| | | | PTC |
| | | | and hedgehog |
| HMGG/LIM | Translocation | Lipoma | Gene fusions high |
| | t(3:12) | | mobility group |
| | t(12:15) | | HMGI-C (XT- |
| | | | hook) |
| | | | and transcription |
| | | | factor |
| | | | LIM or acidic |
| | | | domain |
| JUN | ASV-17 | | Transcription factor |
| | | | AP-1 with FOS |
| MLL/VHRX + | Translocation/fusion | Acute myeloid | Gene fusion of |
| ELI/MEN | ELL with MLL | leukemia | DNA- |
| | Trithorax-like gene | | binding and methyl |
| | | | transferase MLL |
| | | | with |
| | | | ELI RNA pol II |
| | | | elongation factor |
| MYB | Avian | | DNA binding |
| | myeloblastosis | | |
| | Virus | | |
| MYC | Avian MC29; | Burkitt's lymphoma | DNA binding with |
| | Translocation B- | | MAX partner; |
| | cell | | cyclin |
| | Lymphomas; | | regulation; interact |
| | promoter | | RB?; regulate |
| | Insertion avian | | apoptosis? |
| | leukosis | | |
| | Virus | | |
| N-MYC | Amplified | Neuroblastoma | |
| L-MYC | | Lung cancer | |
| REL | Avian | | NF-κB family |
| | | | transcription factor |
| | Retriculoendothelio | | |
| | sis | | |
| | Virus | | |
| SKI | Avian SKV770 | | Transcription factor |
| | Retrovirus | | |
| VHL | Heritable suppressor | Von Hippel-Landau | Negative regulator |
| | | syndrome | or |
| | | | elongin; |
| | | | transcriptional |
| | | | elongation |
| | | | complex |
| WT-1 | | Wilm's tumor | Transcription factor |

CELL CYCLE/DNA DAMAGE
RESPONSE¹⁰⁻²¹

| | | | |
|---|---|---|---|
| ATM | Hereditary disorder | Ataxia- | Protein/lipid kinase |
| | | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group | |
| | | C (predisposition | |
| | | leukemia | |
| FHIT | Fragile site 3p14.2 | Lung carcinoma | Histidine triad- |
| | | | related |
| | | | diadenosine |
| | | | 5′,3″″- |
| | | | P¹ .p⁴ |
| | | | tetraphosphate |
| | | | asymmetric |
| | | | hydrolase |
| hMLI/MutL | | HNPCC | Mismatch repair; |
| | | | MutL |
| | | | homologue |
| hMSH2/MutS | | HNPCC | Mismatch repair; |
| | | | MutS |
| | | | homologue |
| hPMS1 | | HNPCC | Mismatch repair; |
| | | | MutL |
| | | | homologue |
| hPMS2 | | HNPCC | Mismatch repair; |
| | | | MutL |
| | | | homologue |
| INK4/MTS1 | Adjacent INK-4B at | Candidate MTS1 | p16 CDK inhibitor |
| | 9anti-estrogen | suppressor and | |
| | receptor tyrosine | MLM | |
| | kinase inhibitor; | melanoma gene | |
| | CDK complexes | | |
| INK4B/MTS2 | | Candidate | p15 CDK inhibitor |
| | | suppressor | |
| MDM-2 | Amplified | Sarcoma | Negative regulator |
| | | | p53 |
| p53 | Association with | Mutated > 50% | Transcription factor; |
| | SV40 | human | checkpoint control; |
| | T antigen | tumors, including | apoptosis |
| | | hereditary Li- | |
| | | Fraumeni | |
| | | syndrome | |
| PRAD1/BCL1 | Translocation with | Parathyroid | Cyclin D |
| | Parathyroid | adenoma; | |
| | hormone | B-CLL | |
| | or IgG | | |
| RB | Hereditary | Retinoblastoma; | Interact cyclin/cdk; |
| | Retinoblastoma; | osteosarcoma; | regulate E2F |
| | Association with | breast | transcription factor |
| | many | cancer; other | |
| | DNA virus tumor | sporadic | |
| | Antigens | cancers | |
| XPA | | xeroderma | Excision repair; |
| | | pigmentosum; skin | photo- |
| | | cancer | product |
| | | predisposition | recognition; |
| | | | zinc finger |

V. Pharmaceutical Preparations

Pharmaceutical compositions of the present invention comprise an effective amount of one or more forms of anti-estrogen receptor activity compositions or additional agent dissolved or dispersed in a pharmaceutically acceptable carrier or excipient. In a specific embodiment, the anti-estrogen receptor activity composition further comprises tyrosine kinase inhibitor activity. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one anti-estrogen receptor or anti-estrogen receptor and tyrosine kinase inhibitor form or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers,- binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The anti-estrogen receptor or anti-estrogen receptor and tyrosine kinase inhibitor form may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The anti-estrogen receptor or anti-estrogen receptor and tyrosine kinase inhibitor form may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the anti-estrogen receptor or anti-estrogen receptor and tyrosine kinase inhibitor form is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

VI. Anti-ER Activity Compositions and Anti-ER Tyrosine Kinase Inhibitor Activity Compositions

A skilled artisan is aware, based on the teachings provided herein, how to obtain and test compositions for the prevention, treatment, or prevention and treatment of ER positive cancers, and particularly for ER positive breast cancers. Generally, a compound suspected of having anti-ER activity or suspected of having anti-ER activity and anti tyrosine kinase inhibitor activity is administered to a test system, such as a cell line or animal comprising at least one ER positive cancer cell. In one aspect of the present invention, the ER positive cancer cell is also HER-2/neu negative. The inhibition of proliferation of the ER positive cancer cell in the test system is assayed. A compound which inhibits proliferation of the ER positive cancer cell is useful for the prevention, treatment, or prevention and treatment of an ER positive cancer. Examples include emodin, genistein, RG13022 and any compound that can deplete ER.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

Cell Culture—MCF-7, T47D, and ZR-75-1, which are estrogen receptor positive cell lines, were used. They were maintained in phenol red Dulbecco's modified Eagle's/F12 (GIBCO; Grand Island, N.Y.) medium supplemented with 10% fetal bovine serum, 100 units/ml penicillin, and 100 μg/ml streptomycin. Cells were grown in a humidified incubator at 37° C. under 5% CO₂ in air. For experiments requiring estrogen depleted conditions, cells were incubated in phenol red free Dulbecco's modified Eagle's medium supplemented with 1% charcoal stripped serum for one day before initiation of the experiment. The charcoal stripped serum was prepared as described previously (Russell and Hung, 1992).

Reagents—Estrogen (0.1 μM stock solution) (Sigma Chemical Co.; St. Louis, Mo.) was dissolved in ethanol and stored at −20° C. for up to one month. Emodin, genistein, N-Benzyloxycarbonyl-Ile-Glu(O-t-butyl)-Ala-leucinal (PSI), and carbobenzoxyl-leucinyl-leucinyl-norvalinal-II (MG115) were obtained from Sigma Co (St.Louis, Mo.) and RG13022 (Biomol; Plymouth Meeting, Pa.) were dissolved in DMSO. Chloroquine and EGTA (Sigma Co.; St. Louis, Mo.) were dissolved in PBS. L-[³⁵ S]Methionine was from Amersham (Arlington Heights, Ill.). Anti-bcl-2 antibodies were obtained from PharMingen and Sigma Co. Antiestrogen receptor antibody (SRA 1010) was obtained from StressGene (Vancouver, BC). Antiestrogen estrogens, tamoxifen and 4-hydroxytamoxifen were purchased from Sigma Co. Anti-estrogen receptor (D75) (Miller et al., 1993) and anti-hsp90 (AC88) (Redmond et al., 1989) were obtained from Dr. G. Greene and Dr. David Toft, respectively.

Western Analysis—MCF-7 cells were treated with emodin (40 μM) for different time intervals. The cells were lysed with RIPA buffer (20 mM Na₂PO₄, pH 7.4; 150 mM NaCl; 1% TritonX-100; 1% aprotinin: 1 mM phenylmethylsulfonyl fluoride; 10 mg/ml leupeptin; 100 mM NaF; and 2 mM Na₃VO₄). The protein content was determined against a standard control using the Bio-Rad protein assay kit (Bio-Rad Laboratories; Hercules, Calif.). A total of 80-100 μg of total protein lysates were used for SDS-polyacrylamide gel electrophoresis. The proteins were then transferred to a nitrocellulose membrane. The membranes were blocked for 1 h in 5% non-fat dry milk/ Tween 20 (0.1%, v/v) in PBS (PBS/Tween 20). The membranes were incubated with the primary antibodies, anti-estrogen receptor antibody (1:1000; D75), anti-Rb antibody (Santa Cruz Biotech; Santa Cruz, Calif.), anti-Bcl-2 antibody (PharMingen; San Diego, Calif.), and anti-actin antibody for detection of actin for equal loading at 4° C. overnight. The membranes were then incubated with the HRP-goat anti-rat antibody (1:2500 diluation) (Sigma Co.; St. Louis, Mo.), with the HRP-goat anti-mouse antibody (1:10,000 dilution), or with HRP-goat anti-rabbit antibody (1:10,000) (Jackson ImmunoResearch Laboratories, Inc.; West Grove, Pa.) for about 30 minutes at room temperature. The subsequent detection was performed with enhanced chemiluminescence (ECL) system (Amershan Corp.; Arlington Height, Ill.). For the dose responsiveness, MCF-7 cells were treated with different concentration of emodin for 4 hours. Followoing this, the estrogen receptor protein levels were examined by immunoblotting analysis as described above. For detection of the effect of protease inhibitors on emodin-enhanced depletion of estrogen receptor protein, MCF-7 cells were treated with emodin (40 μM) and the protease inhibitors simultaneously for 2 and 4 hours. The controls were treated with the vehicles (DMSO and PBS) alone. The preparation of total protein lysates and subsequent immunoblotting analysis were performed as described above. The intensity of the protein was quantitated by NIH Image. The results were calculated as the percentage of the controls and normalized with actin.

Thymidine incorporation—Cells were detached by trypsinization. 2000 cells were plated on a 96-well microtiter plate overnight. Following this, the medium was replaced with phenol red free Dulbecco's modified Eagle's medium supplemented with 1% charcoal-stripped serum overnight. The cells were then treated with different concentrations of emodin (0, 10, and 40 μM) with or without simultaneous stimulation of estrogen (10 nM) for 20 hours. A set of cells were treated with tamoxifen (10 μM) to compare the effect with emodin. For the control, DMSO and ethanol were added. Six hours before completion of the experiment, 1 μCi of thymidine was added to each well. The cells were then harvested and the radioactivity was measured.

Metabolic labeling of protein, immunoprecipitation and gel electrophoresis—MCF-7 cells were incubated for 1 h at 37° C. in methionine-free medium containing 5% dialyzed, heat-inactivated FCS, with or without emodin (40 μM). [³⁵S] methionine was added to yield 100 μCi/ml in the medium. Incubation was continued for an additional hour. Cells were then rinsed twice with warm, complete medium and were incubated in complete medium with or without emodin (40 μM) at various intervals. Labeled cells were lysed in RIPA-B lysis buffer. Anti-estrogen receptor antibody (D75) immunoprecipitation was performed from 500 μg total cellular protein overnight at 4° C. 20 μg of rabbit anti-rat antibody was added to the mixture and incubated for 45 minutes at 4° C. Protein A-agarose was added and incubated for 45 minutes at 4° C. The mixture was washed three times with cold PBS. Immunoprecipitates were run on 6% SDS-polyacrylamide gels. The gel was fixed with 10% acetic acid/ 30% methanol for at least an hour and was then put into an enhancing solution for 1 hour. The gel was then dried at 80° C. for at least 6 hours. The radiolabeled proteins were visualized by autoradiography. The intensity of the proteins was quantitated by NIH Image. Results were calculated as the percentage of the controls measured at the start of the chase period.

Co-immunoprecipitation—Cellular protein was prepared by lysing the MCF-7 cells treated with 40 μM of emodin for various intervals in RIPA buffer with addition of 10 mM sodium molybdate. Immunoprecipitation with the monoclonal anti-estrogen receptor antibody SRA1010 (Sressgene, Vancouver, BC) was carried out. Immunoprecipitated proteins were analyzed by SDS-polyacrylamide gel electrophoresis and subsequent immunoblotting with anti-hsp90 antibody (AC88) at 1:250 dilution. The membrane was stripped and reprobed with anti-estrogen receptor antibody (D75) for detection estrogen receptor.

### Example 2

### Emodin Mediated Chemopreventive Activity of Breast Tumor Development in Transgenic Mice

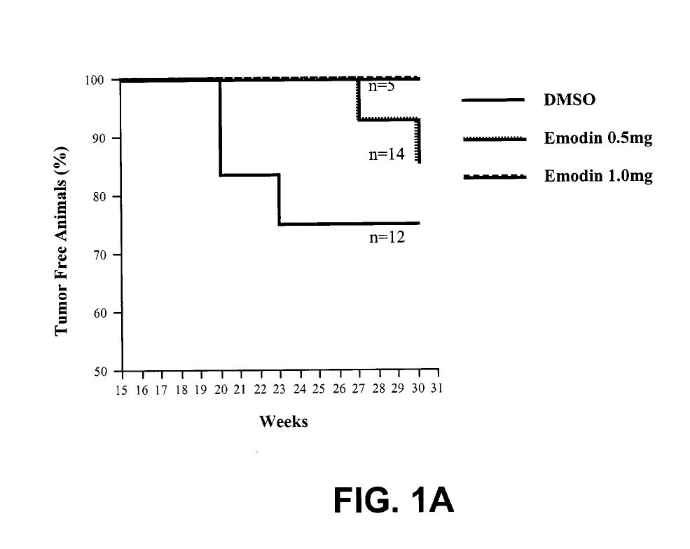

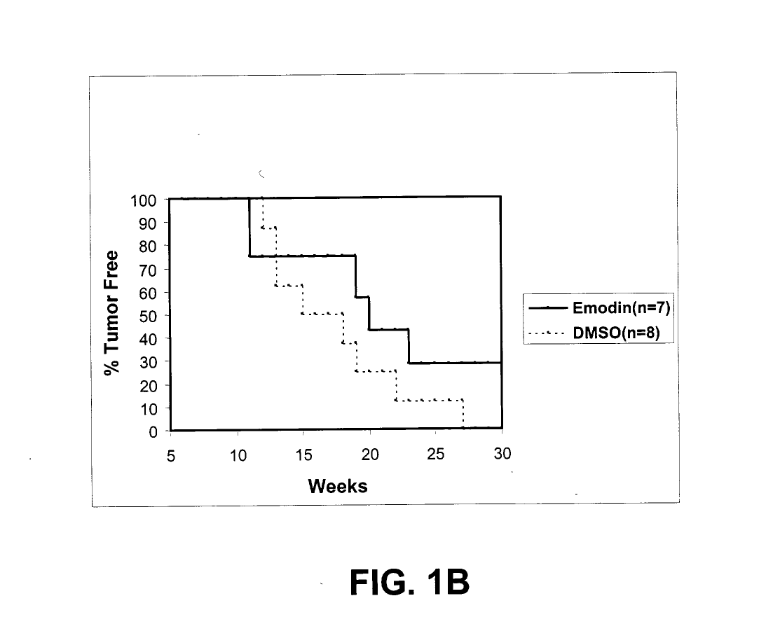

The following example addresses whether a compound may be used as a chemoprevention agent. In a particular embodiment, emodin is tested for its use as a chemopreventive agent. Specifically, a MMTV-neu transgenic mouse model and a MMTV-v-Ha-ras transgenic mouse model were utilized to determine the effects of emodin on the development of breast tumor. In these models, the proto-neu oncogene and v-Ha-ras oncogene, respectively, were under the transcriptional control of the murine mammary tumor virus (MMTV) long terminal repeat (LTR). The transgenic mice develop multiple mammary tumors spontaneously. For FIG. 1A, MMTV-proto-neu female transgenic mice were randomly divided into three groups. At 15 weeks of age, when they did not have tumor symptom yet, mice were treated weekly with emodin 0.5 mg, 1.0 mg or the solvent through intraperitoneal (i.p.) injection. Mice tumor development was monitored weekly. For FIG. 1B, MMTV-v-Ha-ras transgenic mice were administered emodin (n=7) (1 mg/0.2 mL) or DMSO solvent control (n=8) through i.p. injection everyday from six weeks of age.

Thus, FIGS. 1A and 1B provide evidence that emodin associates with chemopreventive activity in mammary tumor development. To further address the molecular mechanism for the prevention activity, the inventors have determined that emodin, like the chemoprevention agent, tamoxifen, can also block ER signaling, albeit through a different mechanism. The detailed studies are described in the following Examples.

### Example 3

### Emodin Inhibits Estrogen-Induced DNA Synthesis in MCF-7 Cells

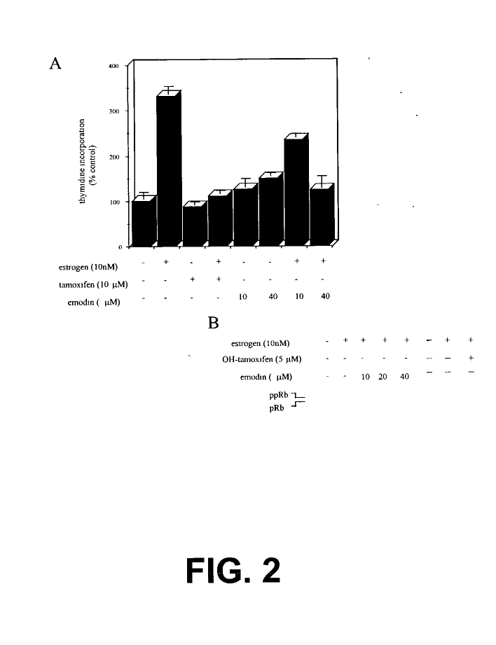

The biological effects of emodin and antiestrogens were compared on the estrogen-induced DNA synthesis and Rb phosphorylation status. MCF-7 cells were treated with different concentrations (10 and 40 μM) of emodin for 20-24 hours with or without estrogen stimulation. In FIG. 2A, MCF-7 cells were treated either tamoxifen or with different concentrations of emodin in the presence or absence of estrogen. The thymidine incorporation rate was then measured. Similar to tamoxifen, emodin can inhibit the estrogen stimulated DNA synthesis as measured by thymidine incorporation assay (FIG. 2A). One of the pathways involved in the estrogen-induced mitogenic activity is phosphorylation of Rb, which inactivates Rb protein function and allows the cells to move from G1 to S phase. Therefore, any correlated changes in Rb phosphorylation status were assayed in these cells by western blotting analysis. After estrogen stimulation, more Rb protein became hyperphosphorylated. FIG. 2B demonstrates western blotting analysis of Rb phosphorylation status in MCF-7 cells after treatment of 4-hydroxytamoxifen or emodin in the presence of estrogen. When cells were treated simultaneously with emodin or 4-hydroxy-tamoxifen, Rb protein remained underphosphorylated (FIG. 2B). Taken together, emodin works similarly as the antiestrogens.

### Example 4

### Depletion of Estrogen Receptor Protein by Emodin

To investigate the mechanisms of an effect mediated by a candidate substance on estrogen-induced function, the candidate substance is tested for an anti-estrogen receptor activity. In general embodiments, the anti-estrogen receptor activity deleteriously affects the activity of the estrogen receptor. The anti-estrogen receptor activity includes, for example, downregulation of the expression of estrogen receptor, an increase in degradation of the estrogen receptor polypeptide, and/or providing an antagonist for the estrogen ligand.

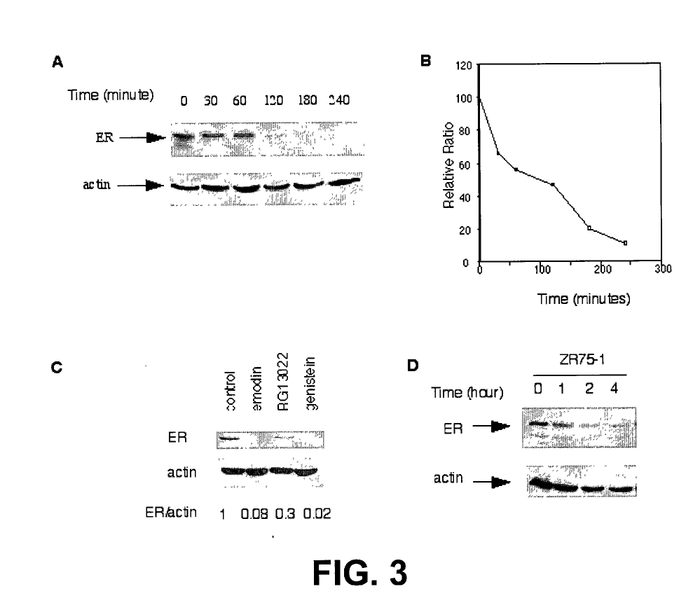

In a specific embodiment, emodin was tested for downregulation of estrogen receptor. MCF-7 cells were treated with 40 μM of emodin at different time intervals, extracted as described in Example 1, followed by examination of the protein levels by western blotting analysis. Estrogen receptor protein levels in MCF-7 cells were measured by immunoblotting with monoclonal antibody D75. The same membrane was stripped and reprobed with anti-β-actin antibody to show the protein loading. As shown in FIG. 3A, emodin could reduce the estrogen receptor protein level rapidly. The proteins were quantitated by NIH Image software and plotted as the percentage control (without emodin) and normalized with actin in FIG. 3B. The estrogen receptor protein level reduced to about 50% of the control after treating the cells with emodin for two hours (FIG. 3B). Then, the IC₅₀ of emodin was measured to induce estrogen receptor depletion by treating the MCF-7 cells with different concentrations of emodin for 4 hours. A dose dependent reduction of estrogen receptor by emodin indicated that the dose required for repressing 50% of estrogen receptor is approximately 20 μM. To examine whether the emodin-induced depletion of estrogen receptor is a general phenomenon, ZR 75-1 and T47D cells, which are known to express estrogen receptor polypeptide, were treated with 40 μM emodin at different time intervals. Emodin could also enhance the depletion of estrogen receptor polypeptide in both ZR 75-1 (FIG. 3D) and T47D cell lines. ZR75-1 estrogen receptor positive breast cancer cell lines were treated with emodin (40 μM) for different time points. Immunoblot analysis for estrogen receptor were performed. Taken together, the results indicate that emodin can rapidly deplete estrogen receptor polypeptide in different estrogen receptor positive cell lines. A skilled artisan recognizes, based on standard knowledge in the art and the data and experiments provided, that any candidate substance may be subjected to similar experiments to assay for downregulation of estrogen receptor and/or depletion of the estrogen receptor polypeptide.

To further investigate whether emodin-induced estrogen receptor depletion is through inhibition of tyrosine kinase(s), two other tyrosine kinase inhibitors, RG13022 and genistein, which have distinct mode of actions from emodin, were used. MCF-7 cells were treated with emodin (40 μM), genistein (100 μM), RG13022 (5 μM) or the solvent for 18 hours. Immunoblot analyses were carried out to detect the estrogen receptor protein level. The same membrane was reprobed with anti-actin antibody for loading control. As shown in FIG. 3C, both RG13022 and genistein also significantly reduced the estrogen receptor polypeptide level. Thus, depletion of estrogen receptor can be demonstrated by three different tyrosine kinase inhibitors, each with distinct mechanisms of action, which strongly suggests that tyrosine kinase pathway is involved in the regulation of the estrogen receptor polypeptide level.

### Example 5

### Decreased Stability of Estrogen Receptor Protein in MCF-7 after Emodin Treatment

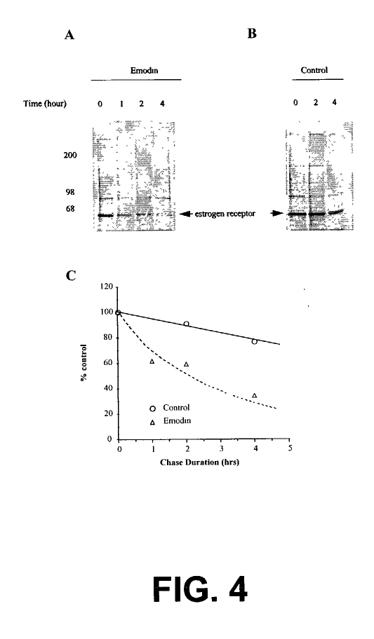

The effect on estrogen receptor was not a consequence of cytotoxicity, because MCF-7 cell morphology, as well as viability as assessed by trypan blue exclusion, were not affected after 4 hour treatment of emodin. The observed decrease in estrogen receptor protein level by emodin could be explained either by diminished protein synthesis or by enhanced protein degradation. To distinguish these two possibilities, a pulse-chase experiment was performed. The cells were labeled with [³⁵S] methionine for 1 hour and chased for 1 to 4 hours in the presence or absence of emodin. Pulse chase experiment was performed to determine the stability of the estrogen receptor proteins after treatment with emodin (see Example 1 for experimental details). In FIG. 4A, MCF-7 cells were treated with emodin at different time intervals. In FIG. 4B, MCF-7 cells were treated with DMSO at various time intervals. In FIG. 4C, proteins were quantitated by NIH Image software and plotted as the percentage of the value at the beginning of the chase.

As shown in FIGS. 4A, 4B, and 4C, the turnover of the newly synthesized estrogen receptor was strikingly enhanced by emodin. The reduction of estrogen receptor level in the pulse chase experiment is consistent with the time course of the decrease of estrogen receptor protein levels as shown in FIG. 3. Taken together, these results demonstrate that the reduction in estrogen receptor protein levels by emodin is not because of diminished protein synthesis but of marked increase in protein degradation.

### Example 6

### Emodin-Induced Estrogen Receptor Degradation involves the Proteasome

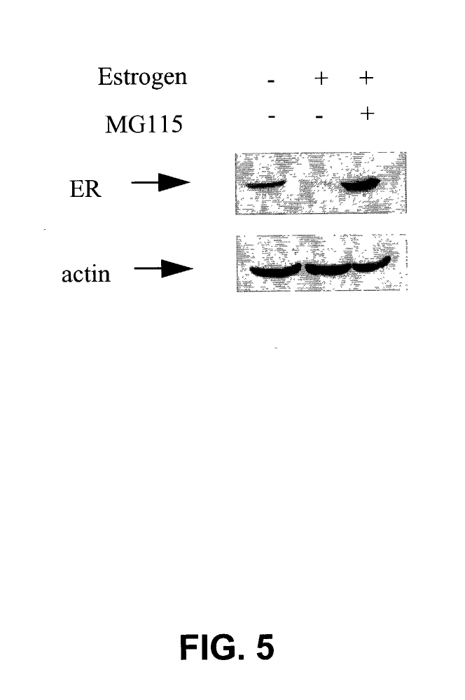

A previous report showed that the rat estrogen receptor was rapidly ubiquitinated after estradiol stimulation (Nirmala and Thampan, 1995), suggesting that 26S proteasome proteolytic pathway may be involved in estrogen receptor degradation. However, no direct experimental evidence has been shown that this degradation pathway is involved. To test whether the estrogen receptor protein can be regulated by the proteasome, MCF-7 cells were stimulated with 10 nM estradiol in the presence or absence of the peptide aldehyde proteasome inhibitor, carbobenzoxyl-leucinyl-leucinyl norvalinal II (MG 115) for 24 hours. For the control, ethanol and DMSO were added. The protein was extracted and quantified by western blot analysis to detect estrogen receptor and actin (for protein loading). As shown in FIG. 5, there was a decrease of estrogen receptor level after estrogen stimulation. However, with the addition of the proteasome inhibitor, the estrogen receptor is protected from degradation. These results indicate that estrogen receptor protein can be regulated by the proteasome pathway.

To further examine whether other proteolytic pathways might be involved in the emodin-enhanced estrogen receptor protein depletion, inhibitors of different proteolytic pathways were added simultaneously with emodin. Then, the estrogen receptor protein levels were detected by western blotting analysis. Chloroquine (100 μM), EGTA (5 mM), MG115 (25 μM), and PSI (25 mM)were added to MCF-7 cells simultaneously with 40 mM emodin at several time intervals. PBS and DMSO were added to the control. The cells were then harvested and the expression levels of estrogen receptor protein were measured by western blotting analysis as described in Example 1.

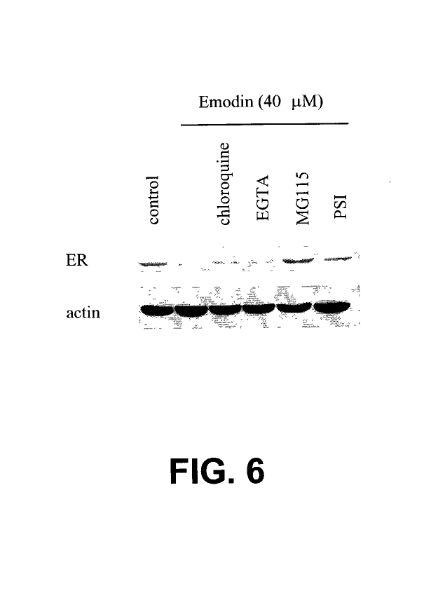

As shown in FIG. 6, the receptor protein remained steady in the control cells but it decreased rapidly in cells treated with 40 μM emodin. The protein levels also decreased in cells treated with either chloroquine, a lysosomal proteolytic inhibitor, or EGTA, a calpain inhibitor. However, in cells treated with PS1 and MG115 resulted in the striking suppression of emodin-enhanced estrogen receptor depletion. Since both PS1 and MG115 are two different kinds of cell permeable proteasome inhibitors, these results further support that the proteasome degradation pathway but not lysosomal or calpains pathways are involved in the emodin-induced estrogen receptor degradation. Taken together, the findings indicate that 26S proteasome proteolytic pathway is involved in the regulation of the estrogen receptor and emodin can enhance the protein degradation through this pathway.

### Example 7

### Increase in Estrogen Receptor-HSP90 Heteromeric Complex Formation after Emodin Treatment

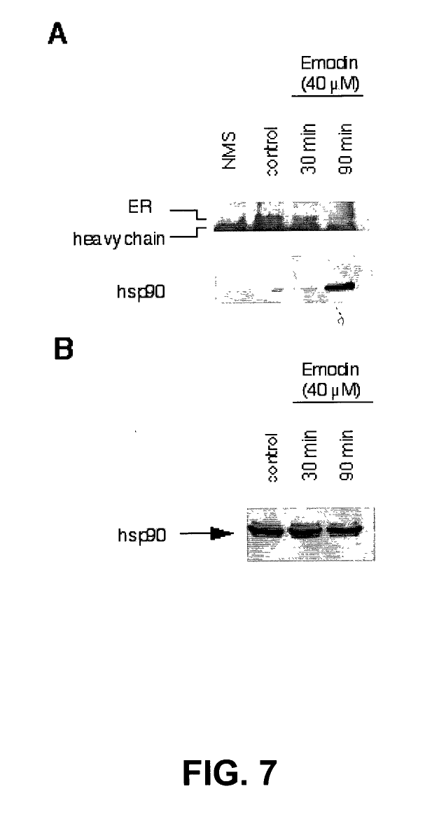

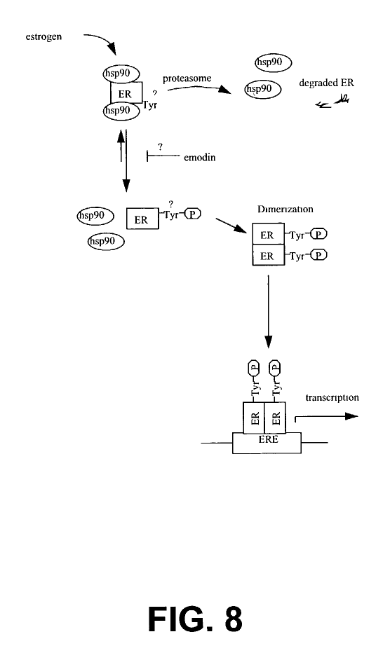

The estrogen receptor is known to form heteromeric complex with two molecules of hsp90 and other proteins. Also, hsp90 is known to play a role in regulation of protein degradation, such as in raf-1. It was therefore examined whether emodin may affect the heteromeric complex formation between hsp90 and the estrogen receptor in association with the receptor degradation. Estrogen receptor immunoprecipitates (by anti-estrogen receptor antibody, SRA1010) from MCF-7 cells were analyzed by SDS/PAGE and immunobloting with anti-hsp90 (AC88). Normal mouse serum (NMS) was used instead of anti-estrogen receptor antibody as a control. In FIG. 7A the membrane was then stripped and reprobed with anti-estrogen receptor (D75) antibodies. The same protein lysates were used to examine the hsp90 protein level by immunoblotting (FIG. 7B). As shown in FIG. 7, there was only small amount of hsp90-estrogen receptor heteromeric complex before emodin treatment. After incubating with 40 μM emodin, the amount of hsp90-bound estrogen receptor markedly increased (FIG. 7A). The cellular levels of hsp90 remained unchanged (FIG. 7B). The results imply that there is apparently only small amount of estrogen receptor bound to hsp90 under steady state conditions. However, emodin inhibits the dissociation of the hsp90-estrogen receptor complex which may lead to the proteasomal degradation of estrogen receptor. Without intending to be bound to any one theory, FIG. 8 illustrates how emodin may induce degradation of the estrogen receptor.

### Example 8

### Additional Transgenic Models

The characterization of the chemoprevention activity of tyrosine kinase inhibitors in vivo is performed in additional transgenic models, similar to what has been described herein, such as MMTV-neuT, and MMTV-c-myc mice, and the like, and the effects are compared between, for example, emodin and other anti-estrogen receptor activity compounds.

### Example 9

### Human Treatment with Anti-Estrogen Receptor Activity Agents in Combination with Anti-Cancer Drugs or Alone

This example describes a protocol to facilitate the treatment of cancer using anti-estrogen receptor activity agents or anti-receptor activity and tyrosine kinase inhibitor agents in combination with anti-cancer drugs. A patient presenting a cancer, in particular an ER positive cancer, may be treated using the following protocol. Patients may, but need not, have received previous chemo- radio- or gene therapeutic treatments. Optimally the patient will exhibit adequate bone marrow function (defined as peripheral absolute granulocyte count of >2,000/mm³ and platelet count of 100,000/mm³, adequate liver function (bilirubin 1.5 mg/dl) and adequate renal function (creatinine 1.5 mg/dl).

Monitoring Estrogen Receptor in Tumors

For tumors that are estrogen receptor positive, the levels of estrogen receptor polypeptide and/or gene expression can be monitored before, during, and after the therapy. The following assay may be used to monitor estrogen receptor polypeptide levels. Sections of 3- to 4 mm thickness of the primary tumors and of the cell block preparations are cut, deparaffinized in xylene, and rehydrated in descending grades (100-70%) of ethanol. Endogenous peroxidase activity is blocked with 3% hydrogen peroxide in methanol. After several washes in distilled water and phosphate-buffered saline, the sections are incubated with a 1:10 dilution of normal horse serum to minimize background staining. This is followed by incubation for 1 hr at room temperature with the primary antibody. The peroxidase staining procedure utilizes ABC Elite Kits (Vector Laboratories; Burlingame, Calif.). The immunostaining reactions are visualized using 3-amino-9-ethylcarbazole as the chromogen. The sections and/or cytospin preparations are stained with toluidine blue and mounted in permount. Positive and negative control immunostains are also prepared.

The sections are reviewed by the pathologist. Two features of the immunoreaction will be recorded using a semi quantitative scale: the relative number of positive cells (0%, <10%, 10-50%, and >50%) and the intensity of the reaction (0-3). The pattern of immunostaining (membranous, cytoplasmic) is recorded separately. A tumor is considered ER positive if any neoplastic cells show increases in estrogen receptor over normal levels. Cytoplasmic staining is considered non-specific. A breast carcinoma known for its strong estrogen receptor staining will be used as a positive control. The quantitative measurement of estrogen receptor immunostaining can be performed using computerized image analysis with the SAMBA 4000 Cell Image Analysis System (Image Products International, Inc., Chantilly, Va.) integrated with a Windows based software. A strong staining tumor tissue section will be used as positive control. The primary antibody will be replaced by an isotype-matched irrelevant antibody to set the negative control threshold, averaging the results from ten fields.

Protocol for the Treatment of Cancer Using Anti-Estrogen Receptor Agents or Anti-Estrogen Receptor and Tyrosine Kinase Inhibitor Agents

A composition of the present invention is typically administered orally or parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. The anti-estrogen receptor agents or anti-estrogen receptor and tyrosine kinase inhibitor agents and/or other tumor suppressing gene products may be delivered to the patient before, after or concurrently with the other anti-cancer agents. A typical treatment course may comprise about six doses delivered over a 7 to 21 day period. Upon election by the clinician, the regimen may be continued six doses every three weeks or on a less frequent (monthly, bimonthly, quarterly, etc.) basis. Of course, these are only exemplary times for treatment, and the skilled practitioner will readily recognize that many other time-courses are possible.

A major challenge in clinical oncology is that many tumor cells are resistant to chemotherapeutic treatment. One goal of the inventors' efforts has been to find ways to improve the efficacy of chemoprevention and/or chemotherapy. In the context of the present invention, anti-estrogen receptor agents or anti-estrogen receptor and tyrosine kinase inhibitor agents can be combined with any of a number of conventional chemotherapeutic regimens.

To kill cancer cells using the methods and compositions described in the present invention, one will generally contact a target cell with an anti-estrogen receptor agent or anti-estrogen receptor and tyrosine kinase inhibitor agent and at least one chemotherapeutic agent (second agent), examples of which are described herein. These compositions will be provided in a combined amount effective to kill or inhibit the proliferation of the cell. This process may involve contacting the cell with anti-estrogen receptor agents or anti-estrogen receptor and tyrosine kinase inhibitor agents and the second agent at the same time. Alternatively, this process may involve contacting the cell with a single composition or pharmacological formulation that includes both agents or by contacting the cell with two distinct compositions or formulations at the same time, wherein one composition includes the anti-estrogen receptor agents or anti-estrogen receptor and tyrosine kinase inhibitor agents and the other includes the second agent.

Alternatively, the anti-estrogen receptor agents or anti-estrogen receptor and tyrosine kinase inhibitor agent administration may precede or follow the delivery of the second agent by intervals ranging from minutes to weeks. In embodiments wherein the anti-estrogen receptor agent or anti-estrogen receptor and tyrosine kinase inhibitor agent and the second compound are applied separately, one would ensure that a significant period of time did not expire between the time of each delivery, such that the second agent and the anti-estrogen receptor agent or anti-estrogen receptor and tyrosine kinase inhibitor agent would still be able to exert an advantageously combined effect on the cancer. In such instances, it is contemplated that one would contact the cell with both agents within about 6 hours to one week of each other and more preferably, within 24-72 hours of each other. In some situations however, it may be desirable to extend the time period for treatment significantly where several days (2, 3, 4, 5, 6, 7 or more) to several weeks (1, 2, 3, 4, 5, 6, 7 or more) lapse between respective administrations.

Regional delivery of anti-estrogen receptor agents or anti-estrogen receptor and tyrosine kinase inhibitor agents will be an efficient method for delivering a therapeutically effective dose to counteract the clinical disease. Likewise, the chemotherapy may be directed to a particular affected region. Alternatively, systemic delivery of either, or both, agent may be appropriate. The therapeutic composition of the present invention is administered to the patient directly at the site of the tumor. This is in essence a topical treatment of the surface of the cancer. The volume of the composition should usually be sufficient to ensure that the entire surface of the tumor is contacted by the anti-estrogen receptor agents or anti-estrogen receptor and tyrosine kinase inhibitor agents and second agent. In one embodiment, administration simply entails injection of the therapeutic composition into the tumor. In another embodiment, a catheter is inserted into the site of the tumor and the cavity may be continuously perfused for a desired period of time.

Clinical responses may be defined by acceptable measure. For example, a complete response may be defined by the disappearance of all measurable disease for at least a month. A partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

Of course, the above-described treatment regimes may be altered in accordance with the knowledge gained from clinical trials such as those described herein. Those of skill in the art will be able to take the information disclosed in this specification and optimize treatment regimes based on the clinical trials described in the specification.

### Example 10

### Clinical Trials of the use of Anti-Estrogen Receptor Agents or Anti-Estrogen Receptor and Tyrosine Kinase Inhibitor Agents in Combination with Anti-Cancer Drugs in Treating Cancer

This example is concerned with the development of human treatment protocols using the anti-estrogen receptor agents or anti-estrogen receptor and tyrosine kinase inhibitor agents in combination with anti-cancer drugs. Anti-estrogen receptor agents or anti-estrogen receptor and tyrosine kinase inhibitor agents and anti-cancer drug treatment will be of use in the clinical prevention, treatment, or prevention and treatment of various cancers in which transformed or cancerous cells play a role, but particularly an ER positive cancer. Such prevention, treatment, or prevention and treatment will be particularly useful tools in anti-tumor therapy, for example, in treating patients with breast cancers that are resistant to conventional chemotherapeutic regimens or in treating patients with a high risk of developing the disease or having a recurrance of the disease.

The various elements of conducting a clinical trial, including patient treatment and monitoring, will be known to those of skill in the art in light of the present disclosure. The following information is being presented as a general guideline for use in establishing anti-estrogen receptor agents or anti-estrogen receptor and tyrosine kinase inhibitor agents in combinations with anti-cancer drugs in clinical trials.

Patients with a high risk of developing ER positive cancer or having a recurrance of ER positive cancer, such as ER positive breast cancer, or who have advanced, metastatic breast ER positive cancers are chosen for clinical study. In a particular embodiment, the patient will typically have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the anti-estrogen receptor agent or anti-estrogen receptor and tyrosine kinase inhibitor agent and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, p185) and in the cells may be assessed and recorded.

In the same procedure, anti-estrogen receptor agent or anti-estrogen receptor and tyrosine kinase inhibitor agent may be administered alone or in combination with the anti-cancer drug. The administration may be in the pleural/peritoneal cavity, directly into the tumor, or in a systemic manner. The starting dose may be 0.5 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of anti-estrogen receptor agent or anti-estrogen receptor and tyrosine kinase inhibitor agent and the lot of anti-cancer drug exceed 5 EU/kg for any given patient.

The anti-estrogen receptor agent or anti-estrogen receptor and tyrosine kinase inhibitor agent and anti-cancer drug combination may be administered over a short infusion time or at a steady rate of infusion over a 7 to 21 day period. The anti-estrogen receptor agent or anti-estrogen receptor and tyrosine kinase inhibitor agent infusion may be administered alone or in combination with the anti-cancer drug. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of anti-estrogen receptor agent or anti-estrogen receptor and tyrosine kinase inhibitor agent in combination with an anti-cancer drug will be administered to groups of patients until approximately 60% of patients show unacceptable Grade III or IV toxicity in any category. Doses that are 2/3 of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored (e.g. CEA, CA 15-3, p185 for breast cancer, and CA 125, p185 for ovarian cancer)

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, p185) and in the cells (p185) may be assessed. For an example of an evaluation profile, see Table 3. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. A urinalysis may be performed every 4 weeks.

Clinical responses may be defined by acceptable measure. For example, a complete response may be defined by the disappearance of all measurable disease for at least a month. A partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

TABLE 3
EVALUATIONS BEFORE AND DURING THERAPY

| | PRE- | TWICE | WEEK- | EVERY 4 | EVERY 8 |
|---|---|---|---|---|---|
| EVALUATIONS | STUDY | WEEKLY | LY | WEEKS | WEEKS |
| History | X | | | X | |
| Physical | X | | | X | |
| Tumor | X | | | X | |
| Measurements | | | | | |
| CBC | X | X¹ | X | | |
| Differential | X | X¹ | X | | |
| Platelet Count | X | X¹ | X | | |
| SMA12-100 | X | | X | | |
| (SGPT, Alkaline | | | | | |
| Phosphatase, | | | | | |
| Bilirubin, | | | | | |
| Alb/Total Protein | | | | | |
| Coagulation | X | | | X | |
| Profile | | | | | |
| Serum Tumor | X | | | X³ | |
| markers (CEA, | | | | | |
| CA15-3, CA-125, | | | | | |
| Her-2/neu) | | | | | |
| Urinalysis | X | | | X | |
| X-rays: | | | | | |
| Chest | X | | X⁴ | | |
| others | X | | | | X |
| Pleural/Peritoneal | X | | X⁵ | X | |
| Fluids: | | | | | |
| (cellularity, | | | | | |
| cytology, LDH, | | | | | |
| tumor markers, | | | | | |
| E1A, HER-1/neu) | | | | | |
| Spirometry and | X | | | X⁶ | X⁶ |
| DLCO X⁶ | | | | | |

¹For the first 4 weeks, then weekly, if no myelosuppression is observed.
²As indicated by the patient's condition.
³Repeated every 4 weeks if initially abnormal.
⁴For patients with pleural effusion, chest X-rays may be performed at 72 hours after first dose, then prior to each treatment administration.
⁵Fluids may be assessed 72 hours after the first dose, weekly for the first two courses and then every 4 weeks thereafter.
⁶Four and eight weeks after initiation of therapy.

While the compositions and methods of this invention have been described in terms of preferred embodiments, it will be apparent to those of skill in the art that variations may be applied to the compositions, methods and in the steps or in the sequence of steps of the methods described herein without departing from the concept, spirit and scope of the invention. More specifically, it will be apparent that certain agents which are both chemically and physically related may be substituted for the agents described herein while the same or similar results would be achieved. All such similar substitutes and modifications apparent to those skilled in the art are deemed to be within the spirit, scope and concept of the invention as defined by the appended claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PATENTS

U.S. Pat. No. 4,554,101

U.S. Pat. No. 5,399,346

U.S. Pat. No. 5,641,484

U.S. Pat. No. 5,643,567

U.S. Pat. No. 5,651,964

U.S. Pat. No. 5,814,315

U.S. Pat. No. 6,172,212

U.S. Pat. No. 6,197,754

PCT/US97/01686

EPO 0273085

### PUBLICATIONS

Al-Obeidi, F. A. and Lam, K. S. (2000) Oncogene 19, 5690-5701

Alfaro-Lopez, J., Yuan, W., Phan, B., Kamath, J., Lou, Q., Lam, K. S. and Hurby, V. J. (1998) J. Med. Chem. 41, 2252-2260

Arap et al., 1995

Arnold, S. F., and Notides, A. C. (1995) Proc. Natl. Acad. Sci. USA 92, 7475-7479

Arnold, S. F., Oboum, J. D., Jaffe, H., and Notides, A. C. (1995) Mol. Endocrinol. 9, 24-33

Arnold, S. F., Vorojeikina, D. P., and Notides, A. C. (1995) J. Biol. Chem. 270, 30205-30212

Aumais, J. P., Lee, H. S., Lin, R., and White, J. H. (1997) J. Biol. Chem. 272, 12229-12235

Baichwal V R, Sugden B. Latency comes of age for herpesviruses. Cell. (1988) 52(6):787-9.

Bakhshi A, Jensen J P, Goldman P, Wright J J, McBride O W, Epstein A L, Korsmeyer S J. Cloning the chromosomal breakpoint of t(14;18) human lymphomas: clustering around JH on chromosome 14 and near a transcriptional unit on 18. Cell. (1985) 41(3):899-906.

Baselga, J., Pfister, D., Cooper, M. R., Cohen, R., Burtness, B., Bos, M., D'Andrea, G., Seidman, A., Norton, L., Gunnett, K., Falcey, J., Anderson, V., Waksal, H., and Mendelsohn, J. (1998) Cancer Res. 58, 2825-2831

Baselga, J., Tripathy, D., Mendelsohn, J., Baughman, S., Benz, C.C., Dantis, L., Sklarin, N. T., Seidman, A. D., Hudis, C. a., Moore, J., Rosen, P. P., Twaddell, T., Henderson, I. C. and Norton, L. (1996) J. Clin. Oncol., 14, 737-744

Baselga J. Clinical trials of single-agent trastuzumab (Herceptin). Semin Oncol. (2000) 27(5 Suppl 9):20-6.

Benvenisty N, Reshef L. Direct introduction of genes into rats and expression of the genes. Proc Natl Acad Sci U S A. (1986) 83(24):9551-5.

Blackwood M A, Weber B L. (1998) Journal of Clinical Oncology 16(5): 1969-1977.

Bocker W. Preneoplasia of the breast. Verh Dtsch Ges Pathol. (1997) 81:502-13.

Bradlow, H. L., Sepkovic, D. W., Telang, N. T., Osborne, M. P. (1995) 768, 180-200

Braunwalder, A. F., Yarwood, D. R., Hall, T., Missbach, M., Lipson, K. E. and Sills, M. A. Anal. Biochem., 234, 23-26

Braunwalder, A. F., Yarwood, D. R., Sills, M. A. and Lipson, K. E. (1996) Anal. Biochem., 238, 159-164

Burke T R Jr, Lim B, Marquez V E, Li Z H, Bolen J B, Stefanova I, Horak I D. Bicyclic compounds as ring-constrained inhibitors of protein-tyrosine kinase p561ck. J Med Chem. (1993) 36(4):425-32.

Caldas C, Hahn S A, da Costa L T, Redston M S, Schutte M, Seymour A B, Weinstein C L, Hruban R H, Yeo C J, Kern S E. Frequent somatic mutations and homozygous deletions of the p16 (MTS1) gene in pancreatic adenocarcinoma. Nat Genet. 1994 Sep;8(1):27-32.

Cardiff R D, Wellings S R, Faulkin L J. Biology of breast preneoplasia. Cancer. (1977) 39(6 Suppl):2734-46.

Cassinell, G., Lanzi, C., Pensa, T., Gambetta, R. A., Nasini, G., Cuccuru, G., Cassinia, M., Pratesi, G., Polizzi, D., Tortoreto, M., and Zunino, P. (2000) Biochem. Pharmacol., 59, 1539-1547

Chan T C, Chang C J, Koonchanok N M, Geahlen R L. Selective inhibition of the growth of ras-transformed human bronchial epithelial cells by emodin, a protein-tyrosine kinase inhibitor. Biochem Biophys Res Commun. (1993) 193(3):1152-8.

Chambraud, B., Berry, M., Redeuilh, G., Chambon, P., and Baulieu, E. E. (1990) J. Biol. Chem. 265, 20686-20691

Chang et al., 1991

Chen C, Okayama H. High-efficiency transformation of mammalian cells by plasmid DNA. Mol Cell Biol. (1987) 7(8):2745-52.

Cheng J Q, Jhanwar S C, Klein W M, Bell D W, Lee W C, Altomare D A, Nobori T, Olopade O I, Buckler A J, Testa J R. p16 alterations and deletion mapping of 9p21-p22 in malignant mesothelioma. Cancer Res. (1994) 54(21):5547-51.

Cleary M L, Smith S D, Sklar J. Cloning and structural analysis of cDNAs for bcl-2 and a hybrid bcl-2/immunoglobulin transcript resulting from the t(14;18) translocation. Cell. (1986) 47(1):19-28.

Cleary M L, Sklar J. Formation of a hybrid bcl-2/immunoglobulin transcript as a result of t(14;18) chromosomal translocation. Hamatol Bluttransfus. (1987) 31:314-9.

Clemens, J. A., Bennett, D. R., Black, L. J., Jones, C. D. (1983) Life Sci. 32, 2869-2875

Coffin J M. Molecular mechanisms of nucleic acid integration. J Med Virol. (1990) 31(1):43-9.

Couch et al., 1963

Coupar B E, Andrew M E, Boyle D B. A general method for the construction of recombinant vaccinia viruses expressing multiple foreign genes. Gene. (1988) 68(1): 1-10.

Coussens L, Yang-Feng T L, Liao Y C, Chen E, Gray A, McGrath J, Seeburg P H, Libermann T A, Schlessinger J, Francke U, et al. Tyrosine kinase receptor with extensive homology to EGF receptor shares chromosomal location with neu oncogene. Science. (1985) 230(4730): 1132-9.

Culver K W, Ram Z, Wallbridge S, Ishii H, Oldfield E H, Blaese R M. In vivo gene transfer with retroviral vector-producer cells for treatment of experimental brain tumors. Science. (1992) 256(5063):1550-2.

Cummings, S. R., Eckert, S., Krueger, K. A. et la. (1999) JAMA 281, 2189-2197

Di Domenico, M., Castoria, G., Bilancio, A., Migliaccio, A., and Auricchio, F. (1996) Cancer Research 56:4516-4521

Diehl, J. A., Zindy, F., and Sherr, C. J. (1997) Genes & Development 11, 957-972

Dubensky T W, Campbell B A, Villarreal L P. Direct transfection of viral and plasmid DNA into the liver or spleen of mice. Proc Natl Acad Sci U S A. (1984) 81(23):7529-33.

Early Breast Cancer Trialists Collaborative Group (1998) Lancet 351, 1451-1467

Fechheimer M, Boylan J F, Parker S, Sisken J E, Patel G L, Zimmer S G. Transfection of mammalian cells with plasmid DNA by scrape loading and sonication loading. Proc Natl Acad Sci U S A. (1987) 84(23):8463-7.

Feramisco J R, Krebs E G. Inhibition of cyclic AMP-dependent protein kinase by analogues of a synthetic peptide substrate. J Biol Chem. (1978) 253(24):8968-71.

Ferkol T, Kaetzel C S, Davis P B. Gene transfer into respiratory epithelial cells by targeting the polymeric immunoglobulin receptor. J. Clin Invest. (1993) 92(5):2394-400.

Fisher, B., Constantino, J. P., Wickerham, D. L., et al. (1998) J. Natl. Cancer Inst. 90, 1371-1388

Ford D, Easton D F, Bishop DT, et al. (1994) Lancet 343(8899): 692-695, 1994.

Ford S L, Reinhart A J, Lukyanenko Y, Hutson J C, Stocco D M. Pregnenolone synthesis in immature rat Sertoli cells. Mol Cell Endocrinol. (1999) 157(1-2):87-94.

Fraley R T, Formari C S, Kaplan S. Entrapment of a bacterial plasmid in phospholipid vesicles: potential for gene transfer. Proc Natl Acad Sci U S A. (1979) 76(7):3348-52.

Frank T S, Manley S A, Olopade O I, et al. (1998) Journal of Clinical Oncology 16(7): 2417-2425.

Friedmann T, Xu L, Wolff J, Yee J K, Miyanohara A. Retrovirus vector-mediated gene transfer into hepatocytes. Mol Biol Med. (1989) 6(2):117-25.

Fry, D. W., McMichael, A., Singh, J., Dorusin, E. M. and McNamara, D. J. (1994) Peptides, 15, 951-957

Fuqua S A, Wiltschke C, Zhang Q X, Borg A, Castles C G, Friedrichs W E, Hopp T, Hilsenbeck S, Mohsin S, O'Connell P, Allred D C. (2000) Cancer Res 60(15), 4026-9

Gao F H, Abee T, Konings W N. Mechanism of action of the peptide antibiotic nisin in liposomes and cytochrome c oxidase-containing proteoliposomes. Appl Environ Microbiol. (1991) 57(8):2164-70.

Ghosh P C, Bachhawat B K. Targeting of liposomes to hepatocytes. Targeted Diagn Ther. (1991) 4:87-103.

Gomez-Foix A M, Coats W S, Baque S, Alam T, Gerard R D, Newgard C B. Adenovirus-mediated transfer of the muscle glycogen phosphorylase gene into hepatocytes confers altered regulation of glycogen metabolism. J Biol Chem. (1992) 267(35):25129-34.

Gopal T V. Gene transfer method for transient gene expression, stable transformation, and cotransformation of suspension cell cultures. Mol Cell Biol. (1985) 5(5):1188-90.

Graham F L, Prevec L. Methods for construction of adenovirus vectors. Mol Biotechnol. (1995) 3(3):207-20.

Graham F L, van der Eb A J. A new technique for the assay of infectivity of human adenovirus 5 DNA. Virology. (1973) 52(2):456-67.

Gribskov M, Burgess R R, Devereux J. PEPPLOT, a protein secondary structure analysis program for the UWGCG sequence analysis software package. Nucleic Acids Res. (1986) 14(1):327-34.

Grunhaus and Horwitz, 1992

Harland R, Weintraub H. Translation of mRNA injected into Xenopus oocytes is specifically inhibited by antisense RNA. J Cell Biol. (1985) 101(3):1094-9.

Hermonat P L, Muzyczka N. Use of adeno-associated virus as a mammalian DNA cloning vector: transduction of neomycin resistance into mammalian tissue culture cells. Proc Natl Acad Sci U S A. (1984) 81(20):6466-70.

Hersdorffer et al., 1990

Hershko, A. (1996) Advances in Experimental Medicine & Biology 389, 221-227

Herz J, Gerard R D. Adenovirus-mediated transfer of low density lipoprotein receptor gene acutely accelerates cholesterol clearance in normal mice. Proc Natl Acad Sci U S A. (1993) 90(7):2812-6.

Hollstein M, Sidransky D, Vogelstein B, Harris C C. p53 mutations in human cancers. Science. (1991) 253(5015):49-53.

Horwich A L, Furtak K, Pugh J, Summers J. Synthesis of hepadnavirus particles that contain replication-defective duck hepatitis B virus genomes in cultured HuH7 cells. J Virol. (1990) 64(2):642-50.

Hudziak R M, Schlessinger J, Ullrich A. Increased expression of the putative growth factor receptor p185HER2 causes transformation and tumorigenesis of NIH 3T3 cells. Proc Natl Acad Sci U S A. (1987) 84(20):7159-63.

Hung M C, Yan D H, Zhao X Y. Amplification of the proto-neu oncogene facilitates oncogenic activation by a single point mutation. Proc Natl Acad Sci U S A. (1989) 86(8):2545-8.

Hussussian C J, Struewing J P, Goldstein A M, Higgins P A, Ally D S, Sheahan M D, Clark W H Jr, Tucker M A, Dracopoli N C. Gernline p16 mutations in familial melanoma. Nat Genet. (1994) 8(1):15-21.

Jacobs T. W., Byrne, C., Colditz, G. et al. (1999) New Engl. J. Med. 340:430-436

Jordan, V. C. (1994) Breast Cancer Res Treat 31(1), 41-52

Ichihara, A., and Tanaka, K. (1995) Molecular Biology Reports 21, 49-52

Jayasuriya, H., Koonchanok, N. M., Geahlen, R. L., McLaughlin, J. L., and Chang, C.-J. (1992) J. Nat. Prod. 55, 696-698

Kamb A, Shattuck-Eidens D, Eeles R, Liu Q, Gruis N A, Ding W, Hussey C, Tran T, Miki Y, Weaver-Feldhaus J, et al. Analysis of the p16 gene (CDKN2) as a candidate for the chromosome 9p melanoma susceptibility locus. Nat Genet. (1994) 8(1):23-6.

Kaneda Y, Iwai K, Uchida T. Introduction and expression of the human insulin gene in adult rat liver. J Biol Chem. (1989) 264(21):12126-9.

Kase, H., Iwahashi, K., Nakanishi, S., Matsuda, Y., Yamada, K., Takahashi, M., Murakata, C., Sato, A. and Kaneko, M. (1987) Biochem. Biophys. Res. Commun. 142, 436-440

Kase Y, Hayakawa T, Ishige A, Aburada M, Komatsu Y. The effects of Hange-shashin-to on the content of prostaglandin E2 and water absorption in the large intestine of rats. Biol Pharm Bull. (1997) 20(9):954-7.

Kato K, Nakanishi M, Kaneda Y, Uchida T, Okada Y. Expression of hepatitis B virus surface antigen in adult rat liver. Co-introduction of DNA and nuclear protein by a simplified liposome method. J Biol Chem. (1991) 266(6):3361-4.

Kato, S., Endoh, H., Masuhiro, Y., Kitamoto, T., Uchiyama, S., Sasaki, H., Masushige, S., Gotoh, Y., Nishida, E., Kawashima, H., Metzger, D., and Chambon, P. (1995) Science 270, 1491-1494

Kern J A, Schwartz D A, Nordberg J E, Weiner D B, Greene M I, Torney L, Robinson R A. p185neu expression in human lung adenocarcinomas predicts shortened survival. Cancer Res. (1990) 50(16):5184-7.

Kerr J F, Wyllie A H, Currie A R. Apoptosis: a basic biological phenomenon with wide-ranging implications in tissue kinetics. Br J Cancer. (1972) 26(4):239-57.

Klein et al., 1987

Kupchan S M, and Karim A. Tumor inhibitors. 114. Aloe emodin: antileukemic principle isolated from Rhamnus frangula L. Lloydia. (1976) 39(4):223-4.

Kyte J, Doolittle R F. A simple method for displaying the hydropathic character of a protein. J Mol Biol. (1982)157(1):105-32.

Lakhani S R. The pathology of familial breast cancer Morphological aspects. Breast Cancer Res. (1999) 1(1):31-5.

Lam, K. S., Wu, J. and Lou, Z. (1995) Int. J. Pept. Protein Res., 45, 587-592

Le Gal La Salle G, Robert J J, Berrard S, Ridoux V, Stratford-Perricaudet L D, Perricaudet M, Mallet J. An adenovirus vector for gene transfer into neurons and glia in the brain. Science. (1993) 12;259(5097):988-90.

Lee, T. R. and Lawrence, D. S. (2000) J. Med. Cehm. 43, 1173-1179

Levitt, M. L., and Koty, P. P. (1999) Investigational New Drugs 17, 213-226

Lishman S C, Lakhani S R. Atypical lobular hyperplasia and lobular carcinoma in situ: surgical and molecular pathology. Histopathology. (1999) 35(3):195-200.

Lou, Q., Leftwich, M. and Lam, K. S. (1996) Bioorg. Med. Chem. 4, 677-682

Lou, Q., Leftwitch, M., McKay, R. T., Salmon, S. E., Rychestsky, L. and Kam, K. S. (1997) Cancer Res., 57, 1877-1881

Macleod K, Leprince D, Stehelin D. The ets gene family. Trends Biochem Sci. (1992) 17(7):251-6.

Mann J R, Corkery J J, Fisher H J, Cameron A H, Mayerova A, Wolf U, Kennaugh A A, Woolley V. The X linked recessive form of XY gonadal dysgenesis with a high incidence of gonadal germ cell tumours: clinical and genetic studies. J Med Genet. (1983) 20(4):264-70.

Marcus, J. N., Watson, P., Page, D. L. et al. (1996) Cancer 77, 697-709

Markowitz D, Goff S, Bank A. A safe packaging line for gene transfer: separating viral genes on two different plasmids. J Virol. (1988) 62(4):1120-4.

Migliaccio, A., Piccolo, D., Castoria, G., Di Domenico, M., Bilancio, A., Lombardi, M., Gong, W., Beato, M., and Auricchio, F. (1998) EMBO J. 17, 2008-2018

Miki Y, Swensen J, Shattuck-Eidens D, et al. (1994) Science 266(5182): 66-71.

Miller, P., DiOrio, C., Moyer, M., Schnur, R. C., Brushin, A., Cullen, W., and Moyer, J. D. (1994) Cancer Res. 54, 2724-2730

Miller, P., Schnur, R. C., Barbacci, E., Moyer, M. P., and Moyer, J. D. (1994) Biochem. Biophys. Res. Commun. 201, 1313-1319

Miller, R. T., Hapke, M. R., and Greene, G. L. (1993) Cancer 71, 3541-6

Mimnaugh, E. G., Chavany, C., and Neckers, L. (1996) J. Biol. Chem. 271, 22796-22801

Monti, E. and Sinha, B. K. (1994) Anticancer Research 14:1221-1226

Mori et al., 1994

Moon et al., 1979

Muller W J, Sinn E, Pattengale P K, Wallace R, Leder P. Single-step induction of mammary adenocarcinoma in transgenic mice bearing the activated c-neu oncogene. Cell. (1988) 54(1):105-15.

Muthuswamy S K, Siegel P M, Dankort D L, Webster M A, Muller W J. Mammary tumors expressing the neu proto-oncogene possess elevated c-Src tyrosine kinase activity. Mol Cell Biol. (1994) 14(1):735-43.

Nabel E G, Plautz G, Nabel G J. Site-specific gene expression in vivo by direct gene transfer into the arterial wall. Science. (1990) 249(4974):1285-8.

Nakagawa, H., Yamamoto, D., Kiyozuka, Y., Tsuta, K., Uemura, Y., Hioki, K., Tsutsui, Y., Tsubura, A. (2000) J Cancer Res Clin Oncol 126:448-454

Needleman S B, Wunsch C D. A general method applicable to the search for similarities in the amino acid sequence of two proteins. J Mol Biol. (1970) 48(3):443-53.

Nicolas J F, Rubenstein J L. Retroviral vectors. Biotechnology. (1988) 10:493-513.

Nicolau C, Sene C. Liposome-mediated DNA transfer in eukaryotic cells. Dependence of the transfer efficiency upon the type of liposomes used and the host cell cycle stage. Biochim Biophys Acta. (1982) 721(2):185-90.

Nicolau C, Legrand A, Grosse E. Liposomes as carriers for in vivo gene transfer and expression. Methods Enzymol. (1987) 149:157-76.

Nirmala P B, Thampan R V. Ubiquitination of the rat uterine estrogen receptor: dependence on estradiol. Biochem Biophys Res Commun. (1995) 213(1):24-31.

Niu, J. and Lawrence, D. S. (1997a) J. Biol. Chem. 272, 1493-1499

Niu, J. and Lawrence, D. S. (1997b) J. Am. Chem. Soc. 119, 3844-3845

Ninnala, P. B., and Thampan, R. V. (1995) Biochem. Biophys. Res. Commun. 213, 24-31

Nobori T, Miura K, Wu D J, Lois A, Takabayashi K, Carson D A. Deletions of the cyclin-dependent kinase-4 inhibitor gene in multiple human cancers. Nature. (1994) 368(6473):753-6.

Okamoto A, Demetrick D J, Spillare E A, Hagiwara K, Hussain S P, Bennett W P, Forrester K, Gerwin B, Serrano M, Beach D H, et al. Mutations and altered expression of p161NK4 in human cancer. Proc Natl Acad Sci U S A. (1994) 91(23):11045-9.

Osborne, M. P., Hoda, S. (1994) Oncology 8:45-49

Osborne M P, Borgen P I, Wong G Y, Rosen P P, McCormick B. Salvage mastectomy for local and regional recurrence after breast-conserving operation and radiation therapy. Surg Gynecol Obstet. (1992) 174(3):189-94.

Osborne M P, Hoda S A. Current management of lobular carcinoma in situ of the breast. Oncology (Huntingt). (1994) 8(2):45-9; discussion 49, 53-4.

Offit K, Gilewski T, McGuire P, et al. (1996) Lancet 347(9016).

Orlow S J, Zhou B K, Chakraborty A K, Drucker M, Pifko-Hirst S, Pawelek J M. High-molecular-weight forms of tyrosinase and the tyrosinase-related proteins: evidence for a melanogenic complex. J Invest Dermatol. (1994) 103(2):196-201.

Page D L, Dupont W D. Anatomic markers of human premalignancy and risk of breast cancer. Cancer. (1990a) 66(6 Suppl):1326-35.

Page D L, Dupont W D. Premalignant conditions and markers of elevated risk in the breast and their management. Surg Clin North Am. (1990b) 70(4):831-51.

Page D L, Dupont W D. Anatomic indicators (histologic and cytologic) of increased breast cancer risk. Breast Cancer Res Treat. (1993) 28(2):157-66.

Page D L, Jensen R A. Evaluation and management of high risk and premalignant lesions of the breast. World J Surg. (1994) 18(1):32-8.

Page D L, Ellis 10, Elston C W. Histologic grading of breast cancer. Let's do it. Am J Clin Pathol. (1995) 103(2):123-4.

Page et al., 1998

Paskind et al., 1975

Perales J C, Ferkol T, Molas M, Hanson R W. An evaluation of receptor-mediated gene transfer using synthetic DNA-ligand complexes. Eur J Biochem. (1994) 226(2):255-66.

Potter H, Weir L, Leder P. Enhancer-dependent expression of human kappa immunoglobulin genes introduced into mouse pre-B lymphocytes by electroporation. Proc Natl Acad Sci U S A. (1984) 81(22):7161-5.

Powles, T., Eles, R., Ashley, S. et al. 91998) Lancet 352, 98-101

Paolini, R., and Kinet, J. P. (1993) EMBO J. 12, 779-786

Paskind M P, Weinberg R A, Baltimore D. Dependence of Moloney murine leukemia virus production on cell growth. Virology. (1975) 67(1):242-8.

Petrakis and Nagabhushan, K.S. (1987) J. Am.Chem. Soc. 109, 2831-2833

Ragot T, Vincent N, Chafey P, Vigne E, Gilgenkrantz H, Couton D, Cartaud J, Briand P, Kaplan J C, Perricaudet M, et al. Efficient adenovirus-mediated transfer of a human minidystrophin gene to skeletal muscle of mdx mice. Nature. (1993) 361(6413):647-50.

Reddy, K. B., Mangold, G. L., Tandon, A. K., Yoneda, T., Mundy, G. R., Zilberstein, A., and Osborne, C. K. (1992) Cancer Res. 52, 3636-3641

Redmond, T., Sanchez, E. R., Bresnick, E. H., Schlesinger, M. J., Toft, D. O., Pratt, W. B., and Welsh, M. J. (1989) Euro. J. Cell Bio. 50, 66-75

Remington's Pharmaceutical Sciences 15^{th} edition.

Renan M J. Cancer genes: current status, future prospects, and applications in radiotherapy/oncology. Radiother Oncol. (1990) 19(3): 197-218.

Rich D P, Couture L A, Cardoza L M, Guiggio V M, Armentano D, Espino P C, Hehir K, Welsh M J, Smith A E, Gregory R J. Development and analysis of recombinant adenoviruses for gene therapy of cystic fibrosis. Hum Gene Ther. (1993) 4(4):461-76.

Richter-Ruoff, B., Wolf, D. H., and Hochstrasser, M. (1994) FEBS Letters 354, 50-52

Ridgeway, 1988

Rippe R A, Brenner D A, Leffert H L. DNA-mediated gene transfer into adult rat hepatocytes in primary culture. Mol Cell Biol. (1990) 10(2):689-95.

Rosen P P, Groshen S, Kinne D W.(1991) Journal of Clinical Oncology 9(9): 1650-1661.

Rosenfeld M A, Siegfried W, Yoshimura K, Yoneyama K, Fukayama M, Stier L E, Paakko P K, Gilardi P, Stratford-Perricaudet L D, Perricaudet M, et al. Adenovirus-mediated transfer of a recombinant alpha 1-antitrypsin gene to the lung epithelium in vivo. Science. (1991) 252(5004):431-4.

Rosenfeld M A, Yoshimura K, Trapnell B C, Yoneyama K, Rosenthal E R, Dalemans W, Fukayama M, Bargon J, Stier L E, Stratford-Perricaudet L, et al. In vivo transfer of the human cystic fibrosis transmembrane conductance regulator gene to the airway epithelium. Cell. (1992) 68(1):143-55.

Roux P, Jeanteur P, Piechaczyk M. A versatile and potentially general approach to the targeting of specific cell types by retroviruses: application to the infection of human cells by means of major histocompatibility complex class I and class II antigens by mouse ecotropic murine leukemia virus-derived viruses. Proc Natl Acad Sci U S A. (1989) 86(23):9079-83.

Ruggeri, B. A., Miknyoczki, S. J., Singh, J. and Hudkins, R. L. (1999) Curr. Med. Chem. 6, 845-857

Russell, K. S., and Hung, M.-C. (1992) Cancer Res. 52, 6624-6629

Sabbah, M., Radanyi, C., Redeuilh, G., and Baulieu, E. E. (1996) Biochemical J. 314, 205-213

Sambrook et al., 1989

Sanba et al., 1985

Schneider P M, Hung M C, Chiocca S M, Manning J, Zhao X Y, Fang K, Roth J A. Differential expression of the c-erbB-2 gene in human small cell and non-small cell lung cancer. Cancer Res. (1989) 49(18):4968-71.

Schneider, C., Sepp-Lorenzino, L., Nimmesgem, E., Ouerfelli, O., Danishefsky, S., Rosen, N., and Hartl, F. U. (1996) Proc. Natl. Acad. Sci. USA 93, 14536-14541

Schmitz, R., Baumann, G. and Gram, H. (1996) J. Mol. Biol. 260, 664-677

Schwartz et al., 1979

Schwartz, G. F., Solin, L. J., Olivotto, I. A. et al. (1999) Breast J. 2000, 4-12

Seethala, R. and Menzel, R. (1997) Anal. Biochem. 253, 210-218

Segnitz, B., and Gehring, U. (1995) Proc. Natl. Acad. Sci. USA 92, 2179-2183

Serrano M, Hannon G J, Beach D. A new regulatory motif in cell-cycle control causing specific inhibition of cyclin D/CDK4. Nature. (1993) 366(6456):704-7.

Serrano P A, Rodriguez W A, Bennett E L, Rosenzweig M R. Protein kinase inhibitors disrupt memory formation in two chick brain regions. Pharmacol Biochem Behav. (1995) 52(3):547-54.

Schechter A L, Stem D F, Vaidyanathan L, Decker S J, Drebin J A, Greene M I, Weinberg R A. The neu oncogene: an erb-B-related gene encoding a 185,000-Mr tumour antigen. Nature (1984) 312(5994):513-6.

Schneider et al., 1981

Shakespeare, W., Yang, M., Bohacek, R., Cerasoli, F., Stebbins, K., Sundaramoorthi, R., Azimioara, M., Vu, C., Pradeepan, S. et al. (2000) Proc. Natl. Acad. Sci USA 97, 9373-9378

Simpson J F, Gray R, Dressler L G, et al. (2000) Journal of Clinical Oncology 18(10): 2059-2069.

Sistonen L, Holtta E, Lehvaslaiho H, Lehtola L, Alitalo K. Activation of the neu tyrosine kinase induces the fos/jun transcription factor complex, the glucose transporter and omithine decarboxylase. J Cell Biol. (1989) 109(5):1911-9.

Smith T F, Waterman M S, Fitch W M. Comparative biosequence metrics. J Mol Evol. (1981) 18(1):38-46.

Smith, D. F. (1993) Mol. Endocrinol. 7, 1418-1429

Songyang, Z., Carraway, K. L., Eck, M. J., Harrison, S. C., Foldman, R. A. et al. (1995) Nature 373, 534-539

Stebbing, J., Copson, E. and O'Reilly, S. (2000) Cancer Treat. Rev., 287-290

Stoll, 1999

Stratford-Perricaudet L D, Briand P, Perricaudet M. Feasibility of adenovirus-mediated gene transfer in vivo. Bone Marrow Transplant. (1992) 9 Suppl 1:151-2.

Stratford-Perricaudet L D, Levrero M, Chasse J F, Perricaudet M, Briand P. Evaluation of the transfer and expression in mice of an enzyme-encoding gene using a human adenovirus vector. Hum Gene Ther. (1990) 1(3):241-56.

Strous, G. J., van Kerkhof, P., Govers, R., Ciechanover, A., and Schwartz, A. L. (1996) EMBO J. 15, 3806-3812

Teixeira, C., Reed, J. C., and Pratt, M. A. (1995) Cancer Res. 55, 3902-3907

Temin H M. Retroviruses and evolution. Cell Biophys. (1986) 9(1-2):9-16.

Tesarik, J., Garrigosa, L., Mendoza, C. (1999) Steroids 64:22-27

The Breast Cancer Linkage Consortium. (1999) Journal of the National Cancer Institute 91(15): 1310-1316.

Top F H Jr, Buescher E L, Bancroft W H, Russell P K. Immunization with live types 7 and 4 adenovirus vaccines. II. Antibody response and protective effect against acute respiratory disease due to adenovirus type 7. J Infect Dis. (1971) 124(2):155-60.

Tsujimoto Y, Cossman J, Jaffe E, Croce C M. Involvement of the bcl-2 gene in human follicular lymphoma. Science. (1985) 228(4706):1440-3.

Tsujimoto Y, Croce C M. Analysis of the structure, transcripts, and protein products of bcl-2, the gene involved in human follicular lymphoma. Proc Natl Acad Sci U S A. (1986) 83(14):5214-8.

Tsurumi, C., Shimizu, Y., Saeki, M., Kato, S., Demartino, G. N., Slaughter, C. A., Fujimuro, M., Yokosawa, H., Yamasaki, M., Hendil, K. B., Toh-e, A., Tanahashi, N., and Tanaka, K. Eur. J. Biochem. 239, 912-921

Tur-Kaspa R, Teicher L, Levine B J, Skoultchi A I, Shafritz D A. Use of electroporation to introduce biologically active foreign genes into primary rat hepatocytes. Mol Cell Biol. (1986) 6(2):716-8.

Varmus H E, Quintrell N, Ortiz S. Retroviruses as mutagens: insertion and excision of a nontransforming provinis alter expression of a resident transforming provirus. Cell. (1981) 25(1):23-36.

Veronesi, U., Maisonneuve, P., Costa, A. et al. (1998) Lancet 352, 93-97

Veronesi, U., De Palo, G., Marubini, E. et al. (1999) J Natl Cancer Inst. (1999) 91, 1847-1856

Wagner E, Zenke M, Cotten M, Beug H, Birnstiel M L. Transferrin-polycation conjugates as carriers for DNA uptake into cells. Proc Natl Acad Sci U S A. (1990) 87(9):3410-4.

Walsh, D. and Glass, D. B. (1991) Methods Enzymol. 201, 304-316

Wang, T. T., and Phang, J. M. (1995) Cancer Res. 55, 2487-2489

Weinberg R A. Tumor suppressor genes. Science. (1991) 254(5035):1138-46.

Weiner D B, Nordberg J, Robinson R, Nowell P C, Gazdar A, Greene M I, Williams W V, Cohen J A, Kern J A.Expression of the neu gene-encoded protein (P185neu) in human non-small cell carcinomas of the lung. Cancer Res. (1990) 50(2):421-5.

Weis, K. E., Ekena, K., Thomas, J. A., Lazennec, G., and Katzenellenbogen, B. S. (1996) Mol. Endocrinol. 10, 1388-1398

Whitesell, L., and Cook, P. (1996) Mol. Endocrinol. 10, 705-712

Wong T K, Nicolau C, Hofschneider P H. Appearance of beta-lactamase activity in animal cells upon liposome-mediated gene transfer. Gene. (1980) 10(2):87-94.

Wu G Y, Wu C H. Receptor-mediated in vitro gene transformation by a soluble DNA carrier system. J Biol Chem. (1987) 262(10):4429-32.

Wu G Y, Wu C H. Receptor-mediated gene delivery and expression in vivo. J Biol Chem. (1988) 263(29):14621-4.

Wu, J. J., Phan, H., Salmon, S. E. and Lam, K. S. (1996) Letters in Peptide Science 3, 309-316

Wu C, Butz S, Ying Y, Anderson R G. Tyrosine kinase receptors concentrated in caveolae-like domains from neuronal plasma membrane. J Biol Chem. (1997) 272(6):3554-9.

Wu C, Butz S, Ying Y, Anderson R G. Tyrosine kinase receptors concentrated in caveolae-like domains from neuronal plasma membrane. J Biol Chem. (1997) 272(6):3554-9.

Yamamoto T, Ikawa S, Akiyama T, Semba K, Nomura N, Miyajima N, Saito T, Toyoshima K. Similarity of protein encoded by the human c-erb-B-2 gene to epidermal growth factor receptor. Nature (1986) 319(6050):230-4.

Yang et al., 1990

Yehetal., 1988

Yu D H, Hung M C. Expression of activated rat neu oncogene is sufficient to induce experimental metastasis in 3T3 cells. Oncogene. (1991) 6(11):1991-6.

Yu D, Wolf J K, Scanlon M, Price J E, Hung M C Enhanced c-erbB-2/neu expression in human ovarian cancer cells correlates with more severe malignancy that can be suppressed by E1A. Cancer Res. (1993) 53(4):891-8.

Yu D, Wang S S, Dulski K M, Tsai C M, Nicolson G L, Hung M C. c-erbB-2/neu overexpression enhances metastatic potential of human lung cancer cells by induction of metastasis-associated properties. Cancer Res. (1994) 54(12):3260-6.

Yu, H., King, R. W., Peters, J. M., and Kirschner, M. W. (1996) Curr. Bio 6, 455-466

Yuan, C. J., Jakes, S., Elliot, S. and Graves, D. J. (1990) J. Biol. Chem. 265, 16205-16209

Zelenin A V, Alimov A A, Barmintzev V A, Beniumov A O, Zelenina I A, Krasnov A M, Kolesnikov V A. The delivery of foreign genes into fertilized fish eggs using high-velocity microprojectiles. FEBS Lett. (1991) 287(1-2):118-20.

Zhang, L., and Hung, M. C. (1996) Oncogene 12, 571-576

Zhang, L., Chang, C. J., Bacus, S. S., and Hung, M. C. (1995) Cancer Res. 55, 3890-3896 Zhang L, Lau Y K, Xi L, Hong R L, Kim D S, Chen C F, Hortobagyi G N, Chang C, Hung M C. Tyrosine kinase inhibitors, emodin and its derivative repress HER-2/neu-induced cellular transformation and metastasis-associated properties. Oncogene. (1998) 16(22):2855-63.

Zhang, L., Lau, Y.-K., Xi, L., Hong, R. L., Kim, Darrick S H L, Chen, C.-F., Hortobagyi, G. N., Chang, C.-J. and Hung, M.-C. (1999) Oncogene 16:2855-2863

Zhang, L., Lau, Y.-K., Xia, W., Hortobagyi, G. N., and Hung, M.-C. (2000) Clinical Cancer Research 5: 343-353

What is claimed is:

1. A method of preventing development or proliferation of one or more estrogen receptor positive cancer cells in an individual comprising administering to the individual a composition having anti-estrogen receptor activity.

2. The method of claim 1, wherein the composition also has tyrosine kinase inhibitor activity.

3. The method of claim 1, wherein the anti-estrogen receptor activity comprises reducing estrogen receptor levels in the cell.

4. The method of claim 1, wherein the anti-estrogen receptor activity comprises modification of the estrogen receptor in the individual.

5. The method of claim 4, wherein the modification comprises degradation of the estrogen receptor.

6. The method of claim 4, wherein the modification comprises downregulation of expression of an estrogen receptor polynucleotide.

7. The method of claim 1, wherein the composition is emodin, genistein, or RG13022.

8. The method of claim 7, wherein the composition is emodin.

9. The method of claim 1, wherein the cell is in vivo.

10. The method of claim 9, wherein the cell is in an animal.

11. The method of claim 10, wherein the animal is a human.

12. The method of claim 11, wherein the human is at an increased risk for developing breast cancer.

13. The method of claim 12, wherein the human has a typical ductal hyperplasia, a typical lobular hyperplasia, a typical epithelial hyperplasia, unfolded lobules, usual ductal hyperplasia, ductal carcinoma in situ, lobular carcinoma in situ, a defective BRCA1 polynucleotide, a defective BRCA2 polynucleotide, an A908G mutation of an estrogen receptor alpha nucleic acid sequence, a breast cancer family history, or a radial scar.

14. A method of treating an estrogen receptor positive and HER-2/neu negative breast cancer cell comprising contacting the cell with a composition comprising tyrosine kinase inhibitor activity and anti-estrogen receptor activity.

15. The method of claim 14, wherein the composition is emodin, genistein, or RG13022.

16. The method of claim 15, wherein the composition is emodin.

17. The method of claim 14, wherein the cell is in vivo.

18. The method of claim 17, wherein the cell is in an animal.

19. The method of claim 18, wherein the animal is a human.

20. The method of claim 19, wherein the contacting of the cell with the composition is concomitant with or subsequent to administration of a breast cancer therapy to said human.

21. The method of claim 20, wherein the breast cancer therapy is radiation, surgery, chemotherapy, biological therapy, immunotherapy, or gene therapy.

22. The method of claim 21, wherein the surgery is lumpectomy or a mastectomy of at least one breast of the individual.

23. The method of claim 21, wherein the chemotherapy comprises an anthracycline, a taxane, an alkylating agent, a fluoropyrimidine, an antimetabolite, a vinca alkaloid, a platinum, or a combination thereof

24. The method of claim 23, wherein the anthracycline is doxorubicin, epirubicin, liposomal doxorubicin, or mitoxantrone.

25. The method of claim 23, wherein the taxane is paclitaxel or docetaxel.

26. The method of claim 23, wherein the alkylating agent is cyclophosphamide.

27. The method of claim 23, wherein the fluoropyrimidine is capecitabine 40 or 5-fluorouracil.

28. The method of claim 23, wherein the antimetabolite is methotrexate.

29. The method of claim 23, wherein the vinca alkaloid is vinorelbine 41, vinblastine, or vincristine.

30. The method of claim 23, wherein the platinum is carboplatin or cisplatin.

31. The method of claim 21, wherein the chemotherapy is gemcitabine, mitomycin C, or herceptin.

32. A method of screening for a compound comprising tyrosine kinase inhibitor activity and anti-estrogen receptor activity, comprising:
contacting an estrogen receptor positive and HER-2/neu negative breast cancer cell with a candidate substance; and
assaying the candidate substance for tyrosine kinase inhibitor activity and anti-estrogen receptor activity in said cell.

33. The method of claim 32, wherein the cell is in an animal, and wherein the animal is assayed for said tyrosine kinase inhibitor activity and said anti-estrogen receptor activity.

34. The method of claim 32, further comprising placing the compound in a pharmacologically acceptable excipient.

35. The method of claim 34, further comprising using the compound in the pharmacologically acceptable excipient to treat an animal having estrogen receptor positive HER-2/neu negative breast cancer.

36. The method of claim 35, wherein the animal is a human.
